(19) United States
(12) Patent Application Publication
(10) Pub. No.: US 2024/0021271 A1
(43) Pub. Date: Jan. 18, 2024

(54) METHODS AND SYSTEMS FOR PREDICTING AN ORIGIN OF A VARIANT

(71) Applicant: GUARDANT HEALTH, INC., Palo Alto, CA (US)

(72) Inventor: Ross Keating EPPLER, San Jose, CA (US)

(21) Appl. No.: 18/172,734

(22) Filed: Feb. 22, 2023

Related U.S. Application Data
(63) Continuation of application No. PCT/US21/47619, filed on Aug. 25, 2021.
(60) Provisional application No. 63/070,182, filed on Aug. 25, 2020.

(51) Int. Cl. G16B 30/10; G16B 20/20; G16H 50/20

(52) U.S. Cl. CPC G16B 30/10; G16B 20/20; G16H 50/20

(57) ABSTRACT

Provided herein are methods for differentiating tumor and non-tumor (e.g., clonal hematopoiesis of indeterminate potential (CHIP)) origin nucleic acid variants from one another in a test sample obtained from a test subject at least partially using a computer. Other aspects are directed to methods of treating disease in subjects. Yet other aspects include related systems and computer readable media used to differentiating tumor and non-tumor origin nucleic acid variants from one another.

### CROSS REFERENCE TO RELATED PATENT APPLICATION

This application is Continuation of PCT No. PCT/US2021/047619, filed Aug. 25, 2021, which claims the benefit to U.S. Provisional Application No. 63/070,182 filed Aug. 25, 2020, herein incorporated by reference in its entirety.

### BACKGROUND

Liquid biopsy next generation sequencing (NGS) assays are known to observe confounding genomic signal from nucleic acid variants originating from white blood cells. Stem cells in the bone marrow ‘white blood cells’ divide to produce new blood cells, and each time a cell divides, there is a chance that a mistake in DNA replication may occur. The high rate of cell division in stem cells allow for the accumulation of mutations, producing daughter blood cells that share these mutations, even though these cells are non-cancerous. The accumulation of mutations in blood cells is called clonal hematopoiesis of indeterminate potential (CHIP). While it is well understood that variants observed in a specific subset of genes provide the majority of confounding CHIP signal, at present it is difficult to adjudicate whether the variant observed in these genes arises from white blood cell or tumor.

Accordingly, there is a need for methods of differentiating tumor and CHIP origin nucleic acid variants from one another.

### SUMMARY

Disclosed are methods of predicting or determining whether a sample is of cancer or non-cancer origin.

Disclosed are methods comprising determining sequence data of a plurality of sequence fragments associated with a plurality of genomic regions, wherein the sequence data comprises a plurality of sequence reads, wherein the plurality of sequence reads are sequenced from the plurality of sequence fragments from a plurality of samples, wherein each sample of the plurality of samples is labeled as a tumor derived or a non-tumor derived; determining at least one of: epigenetic data or fragmentomic data associated with the plurality of sequence fragments; determining, based on at least a portion of the sequence data and at least a portion of at least one of: the epigenetic data or fragmentomic data, a plurality of features for a predictive model; training, based on a first portion of the sequence data and at least one of: the epigenetic data or fragmentomic data, the predictive model according to the plurality of features; testing, based on a second portion of the sequence data and at least one of: the epigenetic data or fragmentomic data, the predictive model; and outputting, based on the testing, the predictive model.

Disclosed are methods comprising determining, for a subject, sequence data of a plurality of sequence fragments associated with a plurality of genomic regions, wherein the sequence data comprises a plurality of sequence reads, wherein the plurality of sequence reads are sequenced from the plurality of sequence fragments from a sample from the subject; determining at least one of: epigenetic data or fragmentomic data associated with the plurality of sequence fragments; providing, to a trained predictive model, at least a portion of the sequence data and at least a portion of at least one of: the epigenetic data or the fragmentomic data; and determining, based on the predictive model, that the sample is tumor-derived or non-tumor derived.

Disclosed are methods of differentiating tumor and clonal hematopoiesis of indeterminate potential (CHIP) origin nucleic acid variants from one another in a test sample obtained from a test subject at least partially using a computer, the method comprising: identifying, by the computer, test nucleic acid variants in a set of targeted genomic regions from sequence information obtained from nucleic acids in the test sample to produce a set of identified test nucleic acid variants; identifying, by the computer, at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups; matching, by the computer, given test nucleic acid variant-epigenetic signature groups in the set of test nucleic acid variant-epigenetic signature groups with reference nucleic acid variant-epigenetic signature groups corresponding to tumor origin nucleic acid variants or with reference nucleic acid variant-epigenetic signature groups corresponding to CHIP origin nucleic acid variants, thereby differentiating the tumor and the CHIP origin nucleic acid variants from one another in the test sample obtained from the test subject.

Disclosed are methods of treating cancer in a test subject, the method comprising: identifying, by a computer, nucleic acid variants in a set of targeted genomic regions from sequence information obtained from nucleic acids in a test sample obtained from the test subject to produce a set of identified test nucleic acid variants; identifying, by the computer, at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups; using, by the computer, at least one trained classifier to differentiate tumor and clonal hematopoiesis of indeterminate potential (CHIP) origin nucleic acid variants from one another in the set of test nucleic acid variant-epigenetic signature groups to produce a set of differentiated tumor and CHIP origin nucleic acid variants present in the test sample; and administering at least one therapy to the test subject based upon one or more of the differentiated tumor origin nucleic acid variants in the set of differentiated tumor and CHIP origin nucleic acid variants present in the test sample, thereby treating the cancer in the test subject.

Disclosed are methods of treating cancer in a test subject, the method comprising administering at least one therapy to the test subject based upon one or more differentiated tumor origin nucleic acid variants in a set of differentiated tumor and clonal hematopoiesis of indeterminate potential (CHIP) origin nucleic acid variants present in the test sample, wherein the set of differentiated tumor and CHIP origin nucleic acid variants is produced by: identifying, by a computer, nucleic acid variants in a set of targeted genomic regions from sequence information obtained from nucleic acids in a test sample obtained from the test subject to produce a set of identified test nucleic acid variants; identifying, by the computer, at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups; and, using, by the computer, at least one trained classifier to differentiate tumor and CHIP origin nucleic acid variants from one another in the set of test nucleic acid variant-epigenetic signature groups.

Disclosed are methods of generating a trained classifier at least partially using a computer, the method comprising: identifying, by the computer, nucleic acid variants in at least one set of targeted genomic regions from sequence information obtained from nucleic acids in a plurality of reference samples to produce a set of identified reference nucleic acid variants; identifying, by the computer, at least one epigenetic signature corresponding to a given nucleic acid variant for a plurality of the identified reference nucleic acid variants in the set of identified reference nucleic acid variants from epigenetic information obtained from the nucleic acids in the reference samples to produce a set of reference nucleic acid variant-epigenetic signature groups; and, training, by the computer, a machine learning algorithm using at least a portion of the set of reference nucleic acid variant-epigenetic signature groups to create at least one trained classifier that is configured to classify one or more test nucleic acid variant-epigenetic signature groups as comprising tumor and/or clonal hematopoiesis of indeterminate potential (CHIP) origin nucleic acid variants.

Disclosed are methods of generating a trained classifier at least partially using a computer, the method comprising: identifying, by the computer, nucleic acid variants in at least one set of targeted genomic regions from sequence information obtained from nucleic acids in a plurality of reference samples to produce a set of identified reference nucleic acid variants; training, by the computer, a machine learning algorithm using at least a portion of the set of identified reference nucleic acid variants to create at least a first model that is configured to classify nucleic acid variants in the set of targeted genomic regions from sequence information obtained from nucleic acids in a test sample to produce a set of identified test nucleic acid variants; identifying, by the computer, at least one epigenetic signature corresponding to a given nucleic acid variant for a plurality of the reference identified nucleic acid variants in the set of identified reference nucleic acid variants from epigenetic information obtained from the nucleic acids in the reference samples to produce a set of reference epigenetic signatures; training, by the computer, the machine learning algorithm using at least a portion of the set of reference epigenetic signatures to create at least a second model that is configured to differentiate tumor and clonal hematopoiesis of indeterminate potential (CHIP) origin nucleic acid variants from one another in the set of test nucleic acid variant-epigenetic signature groups to produce a set of identified test nucleic acid variants, thereby generating the trained classifier.

In some embodiments, the results of the systems and methods disclosed herein are used as an input to generate a report. The report may be in a paper or electronic format. For example, the determination of whether or not a variant and/or sample is tumor derived or non-tumor derived, as determined by the methods and systems disclosed herein, can be displayed directly in such a report.

The various steps of the methods disclosed herein, or steps carried out by the systems disclosed herein, may be carried out at the same or different times, in the same or different geographical locations, e.g., countries, and/or by the same or different people.

### BRIEF DESCRIPTION OF THE FIGURES

The accompanying drawings, which are incorporated in and constitute a part of this specification, illustrate certain embodiments, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically depicts exemplary method steps of differentiating tumor derived and non-tumor derived nucleic acid variants according to some embodiments.

FIG. 2 shows an example of a system that includes an epigenetic component and a fragmentomic component according to an embodiment of the present disclosure.

FIG. 3 shows a process of sequencing a fragment of cell-free (cf) DNA to obtain a methylation state vector.

FIG. 4 shows example methods for determining an end motif.

FIG. 5 shows example methods for assessing a degree of 5′ overhangs.

FIG. 6 shows an example method for determining methylation levels.

FIG. 7 shows an example method for determining an overhang index.

FIG. 8 is an example block diagram for generating a predictive model.

FIG. 9 is a flowchart illustrating an example training method.

FIG. 10 is an illustration of an exemplary process flow for using a machine learning-based classifier.

FIG. 11 shows an example method.

FIG. 12 shows an example method.

FIG. 13 shows an example method.

FIG. 14 shows an example method.

FIG. 15 shows an example method.

### DEFINITIONS

In order for the present disclosure to be more readily understood, certain terms are first defined below. Additional definitions for the following terms and other terms may be set forth through the specification. If a definition of a term set forth below is inconsistent with a definition in a patent application or issued patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, “adapter” refers to short nucleic acids (e.g., less than about 500, less than about 100 or less than about 50 nucleotides in length) that are typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequencing reads of a given nucleic acid molecule. Adapters of the same or different sequence can be linked to the respective ends of a nucleic acid molecule. In certain embodiments, the same adapter is linked to the respective ends of the nucleic acid molecule except that the nucleic acid tag differs in its sequence. In some embodiments, the adapter is a Y-shaped adapter in which one end is blunt ended or tailed as described herein, for joining to a nucleic acid molecule, which is also blunt ended or tailed with one or more complementary nucleotides. In still other exemplary embodiments, an adapter is a bell-shaped adapter that includes a blunt or tailed end for joining to a nucleic acid molecule to be analyzed. Other exemplary adapters include T-tailed and C-tailed adapters.

Administer: As used herein, “administer” or “administering” a therapeutic agent (e.g., an immunological therapeutic agent, a DNA damage response (DDR) inhibitor (e.g., a poly (ADP-ribose) polymerase (PARP) inhibitor (PARPi)), etc.) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Align: As used herein, “align,” alignment,” and “aligning” in the context of nucleic acids refers to arranging sequences of DNA or RNA to identify regions of similarity. Similarity may be related to functional, structural, and/or evolutionary relationships between the sequences. Alignment of DNA sequences involves alignment of genomic DNA of one sequence to genomic DNA of at least one other sequence. Such alignment may exclude non-genomic DNA, such as a molecular barcode, padding bases, and the like. For example, genomic DNA of a sequence read may be aligned to genomic DNA of a reference DNA sequence, excluding any molecular tag that may be attached to the sequence read.

Allele: As used herein, “allele” or “allelic variant” refers to a specific genetic variant at defined genomic location or locus. An allelic variant is usually presented at a frequency of 50% (0.5) or 100%, depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” in the context of nucleic acids refers to a nucleic acid molecule having a sequence that can serve as an identifier of the molecule (molecular barcode), identifier of the partition (partition barcode) or an identifier of the sample (sample barcode or sample index). For example, individual “barcode” sequences are typically added to DNA fragments during next-generation sequencing (NGS) library preparation so that each read can be identified and sorted before the final data analysis.

Breakpoint: As used herein, “breakpoint” in the context of a nucleic acid fusion molecule or a corresponding sequencing read refers to a terminal nucleotide position at a junction between fused sub-sequences of the nucleic acid fusion or represented in the corresponding sequencing read. For example, a given split sequence read may include a first sub-sequence that is contiguous with, and 5′ to, a second sub-sequence in that split sequence read in which the first sub-sequence maps to a first locus in a reference sequence that is non-contiguous with a second locus in that reference sequence to which the second sub-sequence maps. In this example, the first sub-sequence of the split sequence read includes a breakpoint at its 3′ terminal nucleotide, while the second subsequence of the split sequence read includes a breakpoint at its 5′ terminal nucleotide. In certain applications, breakpoints such as these are referred to as a “breakpoint pair.”

Cancer Type: As used herein, “cancer,” “cancer type” or “tumor type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, KRAS, BRAF, NRAS, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell. In some embodiments, “cell-free nucleic acid” refers to nucleic acids which are not contained within or otherwise bound to a cell at the point of isolation from the subject. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Origin: As used herein, “cellular origin” in the context of cell-free nucleic acids means the cell type from which a given cell-free nucleic acid molecule derives or otherwise originates (e.g., via a apoptotic process, a necrotic process, or the like). In certain embodiments, for example, a given cell-free nucleic acid molecule may originate from a tumor cell (e.g., a cancerous pulmonary cell, etc.) or a non-tumor or normal cell (e.g., a non-cancerous pulmonary cell, etc.).

Classifier: As used herein, “classifier” generally refers to algorithm computer code that receives, as input, test data and produces, as output, a classification of the input data as belonging to one or another class (e.g., having a DNA damage repair deficiency (DDRD) or not having DDRD, tumor DNA or non-tumor DNA).

Clonal Hematopoiesis of Indeterminate Potential: As used herein, “clonal hematopoiesis of indeterminate potential” or “CHIP” refers to hematopoiesis in individuals that involves the expansion of hematopoietic stem cells that comprise one or more somatic mutations (e.g., hematologic cancer-associated mutations and/or non-cancer-associated mutations), but which otherwise lack diagnostic criteria for a hematologic malignancy, such as definitive morphologic evidence of dysplasia. CHIP is a common age-related phenomenon in which hematopoietic stem cells contribute to the formation of a genetically distinct subpopulation of blood cells.

Contiguous Sequence: As used herein, “contiguous sequence” or “contig” refers to a set of overlapping nucleic acid segments that together represent a consensus region of a nucleic acid.

Copy Number Variant: As used herein, “copy number variant,” “CNV,” or “copy number variation” refers to a phenomenon in which sections of the genome are repeated and the number of repeats in the genome varies between individuals in the population under consideration.

Coverage: As used herein, the terms “coverage”, “total molecule count” or “total allele count” are used interchangeably. They refer to the total number of DNA molecules at a particular genomic position in a given sample.

Deoxyribonucleic Acid or Ribonucleic Acid: As used herein, “deoxyribonucleic acid” or “DNA” refers a natural or modified nucleotide which has a hydrogen group at the 2′-position of the sugar moiety. DNA typically includes a chain of nucleotides comprising deoxyribonucleosides that comprise one of four types of nucleobases, namely, adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising ribonucleosides that comprise one of four types of nucleobases, namely, A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Detect: As used herein, “detect,” “detecting,” or “detection” refers to an act of determining the existence or presence of one or more target nucleic acids (e.g., nucleic acids having targeted mutations or other markers) in a sample.

Enriched Sample: As used herein, “enriched sample” refers to a sample that has been enriched for specific regions of interest. The sample can be enriched by amplifying regions of interest or by using single-stranded DNA/RNA probes or double stranded DNA probes that can hybridize to nucleic acid molecules of interest (e.g., SureSelect® probes, Agilent Technologies). In some embodiments, an enriched sample refers to a subset or portion of the processed sample that is enriched, where the subset or portion of the processed sample being enriched contains nucleic acid molecules from a sample of cell-free polynucleotides or polynucleotides.

Epigenetic Information: As used herein, “epigenetic information” in the context of a DNA polymer means one or more epigenetic patterns or signatures exhibited in that polymer.

Epigenetic Locus: As used herein, “epigenetic locus” or “epigenetic site” means a fixed position on a chromosome that exhibits different states or statuses that do not involve changes or alterations in nucleotide sequence. For the avoidance of doubt, a given epigenetic locus can coincide with a given nucleotide position or genomic region that also exhibits genetic or sequence variation (e.g., mutations). For example, a given epigenetic locus may or may not be acetylated, methylated (e.g., modified with 5-methylcytosine (5mC), modified with 5-hydroxymethylcytosine (5hmC), and/or the like), ubiquitylated, phosphorylated, sumoylated, ribosylated, citrullinated, have a histone post-translational modification or other histone variation, and/or the like.

Epigenetic rate: As used herein, “epigenetic rate” refers to the probability, likelihood, or percentage of a given epigenetic feature in a DNA molecule. For example, if the epigenetic feature is methylation, then the epigenetic rate refers to the probability, likelihood, or percentage that a given base (for example: cytosine residue in a CpG) is methylated on a DNA molecule. In some embodiments, the epigenetic rate refers to the percentage of residues (for example: CpG residues) with a given epigenetic feature in a DNA molecule. In some embodiments, the epigenetic rate refers to the percentage of residues (for example: CpG residues) with a given epigenetic feature in molecules aligned to particular genomic position or genomic region.

Epigenetic rate threshold: As used herein, “epigenetic rate threshold” refers to a predetermined threshold of the epigenetic rate, which is used to determine the presence of tumor DNA in a sample. For example, if a particular genomic region is hypermethylated in tumor, then if the epigenetic rate at a particular genomic region is greater than the epigenetic rate threshold, then the patient is classified as having cancer. In another example, if a particular genomic region is hypomethylated in tumor, then if the epigenetic rate at a particular genomic region is lower than the epigenetic rate threshold, then the patient is classified as having cancer. The epigenetic rate threshold can be set so as to accommodate embodiments that comprise hypomethylated genomic regions in tumor and hypermethylated genomic regions in tumor. The epigenetic rate threshold can be determined based on a set of training samples (healthy donors and cancer patients or contrived samples) with known tumor fraction. In some embodiments, the epigenetic rate threshold is applied to epigenetic rates of one or more of the plurality of genomic regions.

Epigenetic Signature: As used herein, “epigenetic signature” means an epigenetic state or status exhibited by one or more epigenetic loci in a given DNA molecule. For example, DNA molecules or cfDNA fragments that comprise a given genomic region or locus (e.g., a CTCF binding region, etc.) may also exhibit epigenetic patterns in which some of those DNA molecules include a certain number of epigenetic loci that are methylated, whereas in other instances corresponding epigenetic loci in other DNA molecules or cfDNA fragments that comprise the same genomic region are unmethylated. “Methylation signature” means an epigenetic signature associated with a methylation state or status exhibited by one or more epigenetic loci in a given DNA molecule.

Fusion Event: As used herein, “fusion event” refers to a fusion between at least two separate genes at a particular location. Example causes of a fusion event include a translocation, interstitial deletion, or chromosomal inversion event.

Gene: As used herein, “gene” refers to any segment of DNA associated with a biological function. Thus, genes include coding sequences and optionally, the regulatory sequences required for their expression. Genes also optionally include non-expressed DNA segments that, for example, form recognition sequences for other proteins.

Genomic Region: As used herein, “genomic region” means a fixed position on, or section of, a chromosome, such as the position of a gene or a genomic marker. Exemplary genomic markers include transcriptional factor binding regions (e.g., CTCF binding regions, etc.), distal regulatory elements (DREs), repetitive elements (e.g., microsatellites, etc.), intron-exon or exon-intron junctions, transcriptional start sites (TSSs), and the like.

Germline Mutation: As used herein, “germline mutation” means a mutation in a germ cell and accordingly, that can be passed on to progeny.

Homozygous Deletion: As used herein, “homozygous deletion” or “biallelic inactivation” refers to a mutation or nucleic acid variant that results in the loss of both alleles of a given gene.

Hemizygous Deletion: As used herein, “hemizygous deletion” or “monoallelic inactivation” refers to a mutation or nucleic acid variant that results in the loss of one of two alleles of a given gene. A “heterozygous deletion” is a hemizygous deletion in which the original or initial two alleles of a given gene were different from one another.

Indel: As used herein, “indel” refers to mutation that involves the insertion or deletion of nucleotide positions in the genome of a subject.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Match: As used herein, “match” means that at least a first value or element is at least approximately equal to at least a second value or element. In certain embodiments, for example, the cellular origin of at least the subset of the DNA molecules from a cfDNA sample is determined when there is at least a substantial or approximate match between a test sample distribution of cfDNA fragment properties and a reference sample distribution of cfDNA fragment properties.

Minor Allele Frequency: As used herein, “minor allele frequency” refers to the frequency at which minor alleles (e.g., not the most common allele) occurs in a given population of nucleic acids, such as a sample obtained from a subject. Genetic variants at a low minor allele frequency typically have a relatively low frequency of presence in a sample.

Mutant Allele Fraction: As used herein, “mutant allele fraction,” or “MAF” refers to the fraction of nucleic acid molecules harboring an allelic alteration or mutation with respect to a reference at a given genomic position in a given sample. MAF is generally expressed as a fraction or percentage. For example, MAF is typically less than about 0.5, 0.1, 0.05, or 0.01 (i.e., less than about 50%, 10%, 5%, or 1%) of all somatic variants or alleles present at a given locus.

Maximum Mutant Allele Fraction: As used herein, “maximum mutant allele fraction,” “maximum MAF,” or “MAX MAF” refers to the maximum or largest MAF of all somatic variants present or observed in a given sample.

Mutation: As used herein, “mutation,” “nucleic acid variant,” “variant,” or “genetic aberration” refers to a variation from a known reference sequence and includes mutations such as, for example, single nucleotide variants (SNVs), copy number variants or variations (CNVs)/aberrations, insertions or deletions (indels), truncation, gene fusions, transversions, translocations, frame shifts, duplications, repeat expansions, and epigenetic variants. A mutation can be a germline or somatic mutation. In some embodiments, a reference sequence for purposes of comparison is a wildtype genomic sequence of the species of the subject providing a test sample, typically the human genome. In certain cases, a mutation or variant is a “tumor-related genetic variant” that causes or at least contributes to oncogenesis.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500, about 100, about 50 or about 10 nucleotides in length), used to label nucleic acid molecules to distinguish nucleic acids from different samples (e.g., representing a sample index), or different nucleic acid molecules in the same sample (e.g., representing a molecular tag), of different types, or which have undergone different processing. Nucleic acid tags can be single stranded, double stranded or at least partially double stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or both ends of the other nucleic acids (e.g., sample nucleic acids to be amplified and/or sequenced). Nucleic acid tags can be decoded to reveal information such as the sample of origin, form or processing of a given nucleic acid. Nucleic acid tags can also be used to enable pooling and/or parallel processing of multiple samples comprising nucleic acids bearing different nucleic acid tags and/or sample indexes in which the nucleic acids are subsequently being deconvoluted by reading the nucleic acid tags. Nucleic acid tags can also be referred to as molecular identifiers or tags, sample identifiers, index tags, and/or barcodes. Additionally or alternatively, nucleic acid tags can be used to distinguish different molecules in the same sample. This includes, for example, uniquely tagging different nucleic acid molecules in a given sample, or non-uniquely tagging such molecules. In the case of non-unique tagging applications, tags with a limited number of different sequences may be used to tag nucleic acid molecules such that different molecules can be distinguished based on, for example, start and/or stop positions where they map to a selected reference genome in combination with at least one nucleic acid tag. Typically, a sufficient number of different nucleic acid tags are used such that there is a low probability (e.g., less than about a 10%, less than about a 5%, less than about a 1%, or less than about a 0.1% chance) that any two molecules will have the same start/stop positions and also have the same nucleic acid tag. Some nucleic acid tags include multiple molecular identifiers to label samples, forms of nucleic acid molecules within a sample, and nucleic acid molecules within a form having the same start and stop positions. Such nucleic acid tags can be referenced using the exemplary form “Ali” in which the uppercase letter indicates a sample type, the Arabic numeral indicates a form of molecule within a sample, and the lowercase Roman numeral indicates a molecule within a form.

Nucleic Acid Variant-Epigenetic Signature Group: As used herein, “nucleic acid variant-epigenetic signature group” refers to nucleic acid variants and epigenetic signatures that correlate with one another (e.g., an epigenetic signature observed in a genomic region that comprises the nucleic acid variant or the like).

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Prevalence: As used herein, “prevalence” in the context of nucleic acid variants refers to the degree, pervasiveness, or frequency with which a given nucleic acid variant is or was observed in a given sample (e.g., a given bodily fluid sample, a given non-bodily fluid sample, etc.) or other population (e.g., a given population of bodily fluid samples, a given population of non-bodily fluid samples, etc.).

Reference Sample: As used herein, “reference sample” or “reference cfDNA sample” refers a sample of known composition and/or having or known to have or lack specific properties (e.g., known nucleic acid variant(s), known cellular origin, known tumor fraction, known coverage, and/or the like) that is analyzed along with or compared to test samples in order to evaluate the accuracy of an analytical procedure. A reference sample dataset typically includes from at least about 25 to at least about 30,000 or more reference samples. In some embodiments, the reference sample dataset includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more reference samples.

Reference Sequence: As used herein, “reference sequence” or “reference genome” refers to a known sequence used for purposes of comparison with experimentally determined sequences. For example, a known sequence can be an entire genome, a chromosome, or any segment thereof. A reference sequence typically includes at least about 20, at least about 50, at least about 100, at least about 200, at least about 250, at least about 300, at least about 350, at least about 400, at least about 450, at least about 500, at least about 1000, at least about 10,000, at least about 100,000, at least about 1,000,000, at least about 10,000,000, at least about 100,000,000, at least about 1,000,000,000, or more nucleotides. A reference sequence can align with a single contiguous sequence of a genome or chromosome or can include non-contiguous segments that align with different regions of a genome or chromosome. Exemplary reference sequences, include, for example, human genomes, such as, hG19 and hG38.

Sample: As used herein, “sample” means any biological sample capable of being analyzed by the methods and/or systems disclosed herein. In certain aspects of the present disclosure, samples are bodily fluid samples, for example, whole blood or fractions thereof, lymphatic fluid, urine, and/or cerebrospinal fluid, among other bodily fluid types from which cell-free (circulating, not contained within or otherwise bound to a cell) nucleic acids are sourced. In certain implementations, bodily fluid samples are plasma samples, which are the fluid portions of whole blood exclusive of cells, such as red and white blood cells. In some implementations, bodily fluid samples are serum samples, that is, plasma lacking fibrinogen. In some aspects of the present disclosure, samples are “non-bodily fluid samples” or “non-plasma samples,” that is, biological samples other than “bodily fluid samples” such as, as cellular and/or tissue samples, from which nucleic acids other than cell-free nucleic acids are sourced.

Sensitivity: As used herein, “sensitivity” in the context of a given assay or method refers to the ability of the assay or method to detect and distinguish between targeted (e.g., nucleic acid variants) and non-targeted analytes.

Sequence fragment: As used herein, “sequence fragment” refers to a nucleic acid molecule or a portion thereof that can vary in length and can carry the sequence information (or sequence data) of the nucleic acid molecule. The sequence information can be derived from sequencing reads obtained from sequencing the sequence fragments.

Sequence read: As used herein, “sequence read” refers to the sequence of nucleotides corresponding to all or a part of a sequence fragment and is generated by a sequencer (for example, a next generation sequencer like, but not limited to, Illumina sequencer).

Sequencing: As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and/or identity of monomer units (e.g., nucleotides, etc.) in that polymer.

Sequence Motif: As used herein, “sequence motif” may refer to a short, recurring pattern of bases in DNA fragments (e.g., cell-free DNA fragments). A sequence motif can occur at an end of a fragment, and thus be part of or include an ending sequence. An “end motif” can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif.

Single Nucleotide Variant: As used herein, “single nucleotide variant” or “SNV” means a mutation or variation in a single nucleotide that occurs at a specific position in the genome.

Somatic Mutation: As used herein, “somatic mutation” means a mutation in a given genome that occurs after conception. Somatic mutations can occur in any cell of the body except germ cells and accordingly, are not passed on to progeny.

Specificity: As used herein, “specificity” in the context of a diagnostic analysis or assay refers to the extent to which the analysis or assay detects an intended target analyte to the exclusion of other components of a given sample.

Status: As used herein, “status” in the context of subjects refers to one or more states of a given subject, such as whether or not the subject has cancer.

Subject: As used herein, “subject” or “test subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” In some embodiments, the subject is a human who has, or is suspected of having cancer. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed with or suspected of having a disease, e.g., a cancer, an auto-immune disease. A “reference subject” refers to a subject known to have or lack specific properties (e.g., known cancer or disease status, known nucleic acid variant(s), known cellular origin, known tumor fraction, known coverage, and/or the like).

Threshold: As used herein, “threshold” refers to a separately determined value used to characterize or classify experimentally determined values. In certain embodiments, for example, “threshold value” refers to a selected value to which a quantitative value is compared in order to determine that a given target nucleic acid variant is absent at a given genetic locus.

Tumor Fraction: As used herein, “tumor fraction” refers to the estimate of the fraction of nucleic acid molecules derived from tumor in a given sample. For example, the tumor fraction of a sample can be a measure derived from the maximum mutant allele frequency (MAX MAF) of the sample or coverage of the sample, or length, epigenetic state, or other properties of the cfDNA fragments in the sample or any other selected feature of the sample. The term “MAX MAF” refers to the maximum or largest MAF of all somatic variants present in a given sample. In some embodiments, the tumor fraction of a sample is equal to the MAX MAF of the sample.

Value: As used herein, “value” or “score” generally refers to an entry in a dataset can be anything that characterizes the feature to which the value refers. This includes, without limitation, numbers, words or phrases, symbols (e.g., + or −) or degrees.

### DETAILED DESCRIPTION

### I. Introduction

Provided herein are methods and systems for differentiating or classifying tumor and non-tumor origin nucleic acid variants in a nucleic acid sample obtained from a test subject. In some aspects, the methods and systems couple somatic sequence data (e.g., somatic genomic data) with epigenetic data. In some aspects, the methods and systems couple sequence data with fragmentomics data. In some aspects, the methods and systems couple sequence data with epigenetic data and fragmentomics data. The epigenetic data and/or fragmentomics data may provide additional genomic signal to aid in determining the origin (e.g., tumor or non-tumor) of a variant in the sequence data. For example, the variant may be the result of clonal hematopoiesis of indeterminate potential (CHIP). In some aspects, the nucleic acid sample can be, but is not limited to, cell-free nucleic acid (cfNA), genomic DNA, or RNA.

In certain embodiments, incorporation of targeted hybridization panels investigating known methylation sites or other epigenetic sites in genes of likely CHIP interference (e.g., DNMT3A, TP53, LRP1B, KRAS, etc.) may be used to contribute to the determination of the origin of the variant in the somatic genomic data.

Essentially any number of genes may be optionally evaluated using the methods and related aspects of the present disclosure. In some embodiments, for example, sets of genes targeted for analysis, as described herein, include at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 100, 1,000, 10,000, or more genes. A non-exhaustive list of genes, one or more of which are optionally selected for evaluation using the methods and related aspects disclosed herein is provided in Table 1.

TABLE 1

| GENE | TUMOR TYPE |
|---|---|
| ALK | LUNG; MELANOMA |
| fusions | |
| ALK | THYROID |
| fusions (anaplastic) | |
| ATM MUTATIONS | PROSTRATE |
| BRAF | LUNG; COLORECTAL |
| V600E | |
| BRAF | GIST |
| mutations | |
| BRAF | MELANOMA |
| V600E/K | |
| BRAF | THYROID |
| V600E (anaplastic) | |
| BRCA1/2 | BREAST; PROSTRATE; |
| germline and somatic | PANCREATIC |
| EGFR | LUNG |
| exon 19 del, L858R and other alterations | |
| EGFR | LUNG |
| exon 20 insertions | |
| EGFR | COLORECTAL |
| mutations | |
| ERBB2 | LUNG |
| exon 20 insertions and other alterations | |
| ERBB2 (HER2) | BREAST; COLORECTAL; |
| amplification | ENDOMETRIAL; |
| | GASTRIC/GASTROESOPHAGEAL |
| ESR1 | BREAST |
| mutations | |
| EXPANDED HRR | PROSTRATE |
| genes | |
| EXTENDED KRAS | COLORECTAL |
| mutations | |
| EXTENDED NRAS | COLORECTAL |
| mutations | |
| FGFR2 | CHOLANGIOCARCINOMA |
| fusions | |
| FGFR2/3 | BLADDER; GIST |
| fusions and other alterations | |
| IDH1 | CHOLANGIOCARCINOMA |
| mutations | |
| KIT | GIST; MELANOMA |
| mutations | |
| KRAS | LUNG |
| G12C | |
| MET | LUNG |
| exon 14 skipping | |
| MET | LUNG |
| amplifications | |
| MSI-HIGH | BREAST; PROSTRATE; |
| | COLORECTAL; ENDOMETRIAL |
| NRAS | MELANOMA |
| mutations | |
| NTRK1/2/3 | LUNG; BREAST; PROSTRATE; |
| fusions | COLORECTAL |
| PDGFRA | GIST |
| mutations | |
| PIK3CA | BREAST |
| mutations | |
| RET | LUNG; THYROID |
| fusions | |
| ROS1 | LUNG; MELANOMA |
| fusions | |
| TMB | LUNG |

Exemplary sets of genes that may be evaluated as described herein to identify patients that are candidates for specific targeted therapies are listed in Table 2.

TABLE 2

| GENE | ASSOCIATED THERAPY |
|---|---|
| ALK | ALECENSA ®, ALUNBRIG ®, ZYKADIA®, |
| fusions | XALKORI ®, LORBRENA ® (40-80%) |
| ATM MUTATIONS | LYNPARZA ® (22-33%) |
| BRAF | TAFINLAR ® + MEKINIST ® (65%) |
| V600E | BRAFTOVI ® + ERBITUX ® (20%) |
| BRCA1/2 | LYNPARZA ®, TALZENNA ® (60-63%) |
| germline and somatic | LYNPARZA ® (22%-33%), RUBRACA® |
| | (44%) |
| EGFR | TAGRISSO ®, TARCEVA ®, GILOTRIF ®, |
| exon 19 del, L858R and other alterations | IRESSA ®, VISIMPRO ® (60-80%) |
| EGFR | RYBREVANT ™ (40%) |
| exon 20 insertions | |
| EGFR | PREDICTS LACK OF RESPONSE TO |
| mutations | ERBITUX ®, VECTIBIX ® |
| ERBB2 | KADCYLA ® (45%), HERCEPTIN ® |
| exon 20 insertions and other alterations | combinations (50%) |
| ERBB2 (HER2) | HERCEPTIN ®, PERJETA ®, KADCYLA®, |
| amplification | (14-80%) |
| | HERCEPTIN ® + PERJETA ® |
| | HERCEPTIN ® + TYKERB ® (27-32%) |
| EXPANDED HRR | LYNPARZA ® (22-33%) |
| genes | |
| EXTENDED KRAS | PREDICTS LACK OF RESPONSE TO |
| mutations | ERBITUX ®, VECTIBIX ® |
| EXTENDED NRAS | PREDICTS LACK OF RESPONSE TO |
| mutations | ERBITUX ®, VECTIBIX ® |
| KRAS | LUMAKRAS ™ (37%) |
| G12C | |
| MET | TABRECTA ™, TEPMETKO ™ (41-68%) |
| exon 14 skipping | |
| MET | XALKORI ® (40%) |
| amplifications | |
| MSI-HIGH | KETRUDA ® (40%) |
| | KEYTRUDA ® (39.6-46%) |
| | KEYTRUDA ®, OPDIVO ® + |
| | YERVOY ® (40-50%) |
| NTRK1/2/3 | VITRAKVI ®, ROZLYTREK ™ (57-75%) |
| fusions | |
| PIK3CA | PIQRAY ® (27%) |
| mutations | |
| RET | RETEVMO ™, GAVRETO™ (55-85%) |
| fusions | |
| ROS1 | XALKORI ®, ROZLYTREK ® (72-78%) |
| fusions | |
| TMB | KEYTRUDA ® (29-37%) |

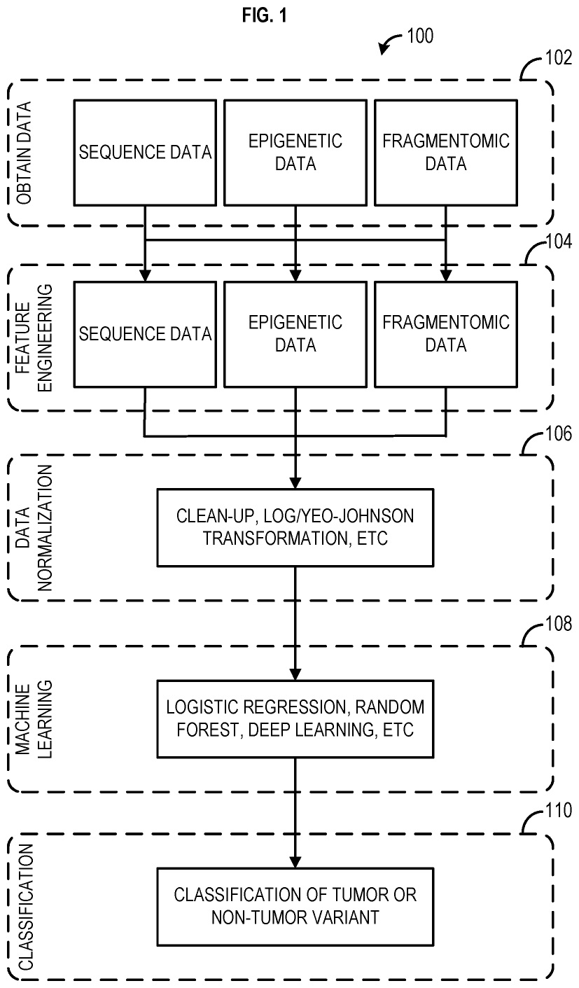

FIG. 1 is a flow chart that schematically depicts an example artificial intelligence (e.g., machine learning) technique for generating a classifier configured for differentiating or classifying tumor and non-tumor origin nucleic acid variants in a cell-free nucleic acid (cfDNA) sample obtained from a test subject. As shown, a method 100, at step 102, may comprise obtaining data, for example, in the form of cancer (e.g., tumor) origin and non-cancer origin sequence data from cell-free nucleic acid (cfDNA) samples of a plurality of subjects. The method 100 may also comprise obtaining epigenetic data and/or fragmentomic data associated with, or otherwise derived from, the sequence data. Sequence data, epigenetic data, and fragmentomic data can all be determined from genomic regions within the cfDNA samples. Epigenetic data may include, for example, information regarding DNA methylation, histone states or modifications, inflammation-mediated cytosine damage products, protein binding, or other molecular states reflected in the nucleic acid fragment analyzed that are not ascertained solely from the nucleotide base sequence, e.g., the methylation status of give base or set bases. Fragmentomic data may include, for example, information regarding fragment size, nucleotide motifs at fragment ends, single-stranded jagged ends, genomic location of center point of the fragment length, genomic locations of fragment endpoints, genomic locations of fragment endpoints and/or any value indicating the endpoints of the fragment. In an embodiment, the origin of the sequence fragments and/or variants in the sequence data may also be associated with the sequence data, the epigenetic data, and/or the fragmentomic data. For example, sequence data, epigenetic data, and fragmentomic data of sequence fragments and/or variants known to be tumor derived may be labeled as tumor derived and sequence data, epigenetic data, and fragmentomic data of sequence fragments and/or variants known to be non-tumor derived may be labeled as non-tumor derived. Moreover, further labels may be assigned, for example, cancer type, tissue type, and the like.

In some embodiments, the methods, and related systems and computer readable media implementations, disclosed herein include identifying sets of DNA molecules or cfDNA fragments from cfDNA samples in which each member cfDNA fragment of a given set comprises a genomic region in common with one another. Essentially any genomic region can be used as long as cfDNA fragments comprising a given genomic region exhibit different properties (e.g., cfDNA fragment lengths, offsets of cfDNA fragment midpoints relative to midpoints of genomic regions comprised by the cfDNA fragment, epigenetic states, and/or the like) between at least two cell or tissue types. In certain embodiments, for example, genomic regions include regions of differential chromatin organization between at least two cell or tissue types. More specifically, fragmentation patterns of DNA molecules in cfDNA samples carries information about the chromatin organization of the cells or tissues from which the cfDNA fragments originate. In particular, DNA fragments released to the bloodstream is often fragmented or cleaved around nucleosomes and/or other DNA bound proteins in the cells or tissues of origin. Further, nucleosome positioning and the location of DNA binding proteins is highly tissue specific and thus is used herein to amplify signal coming from the cells or tissues from which the cfDNA fragments originate (e.g., tumor cells as well as cells in the tumor microenvironment and cells involved in the immune response). In certain embodiments, genomic regions comprise transcriptional factor binding regions, distal regulatory elements (DREs), repetitive elements, intron-exon or exon-intron junctions (splice junctions), transcriptional start sites (TSSs), and/or the like.

A transcription factor (or sequence-specific DNA-binding factor) is a protein that regulates the rate of transcription of genetic information from DNA to messenger RNA, by binding to a specific DNA recognition sequence. Transcription factors are also oftentimes involved in other cellular processes beyond transcriptional regulation. There are thought to be around 2600 transcription factors in the human genome. A transcription factor includes at least one DNA-binding domain (DBD), which binds to a specific recognition sequence of DNA adjacent to the gene that it regulates. Non-limiting examples of transcription factors include CCCTC-binding factor (CTCF or 11-zinc finger protein)(recognition sequence: 5′-CCGCGNGGNGGCAG-3′ (SEQ ID NO: 1)), SP1 (recognition sequence: 5′-GGGCGG-3′), C/EBP (recognition sequence: 5′-ATTGCGCAAT-3′ (SEQ ID NO: 2)), AP-1 (recognition sequence: 5′-TGA(G/C)TCA-3′), c-Myc (recognition sequence: 5′-CACGTG-3′), ATF/CREB (recognition sequence: 5′-TGACGTCA-3′), and Oct-1 (recognition sequence: 5′-ATGCAAAT-3′). The genomic regions used in the methods described herein optionally include one or more of these or any other transcription factor recognition sequences or binding sites. Additional details regarding transcription factors and related recognition sequences are described in, for example, Latchman, “Transcription factors: an overview,” The International Journal of Biochemistry & Cell Biology, 29(12):1305-12 (1997) and Ptashne et al., “Transcriptional activation by recruitment,” Nature, 386(6625):569-77, which are incorporated by reference.

To further illustrate, CTCF is a transcription factor (also known as transcriptional receptor CTCF, 11-zinc finger protein, or CCCTC-binding factor) involved in many cellular processes, including but not limited to, transcription regulation and chromatin organization. Binding of CTCF can be tissue specific and can induce strong nucleosomal organization upstream and downstream of the CTCF binding site. Therefore, perturbation of such nucleosomal organization due to contribution of tissues unique to, for example, plasma cfDNA of cancer patients may be detected and revealed by analyzing the cfDNA fragment (fragmentomics) pattern in and around these sites (CTCF binding regions). Additional details regarding inferring genomic regions, such as CTCF binding sites and related aspects that are adapted for use in performing the methods described herein are disclosed in U.S. Provisional Application No. 62/692,495, filed Jun. 29, 2018, which is incorporated by reference.

Distal regulatory elements (DREs) are involved in transcription regulation and include locus control regions, enhancers, insulators, and silencing elements. Binding sites related to DREs are optionally used as genomic regions in the methods described herein. Additional details regarding DREs are described in, for example, Heintzman et al., “Finding distal regulatory elements in the human genome,” Curr Opin Genet Dev, December; 19(6):541-549 (2009), which is incorporated by reference.

Repetitive elements are recurring patterns of nucleotides that are present in multiple copies throughout a given genome and/or a population of genomes. Non-limiting examples of repetitive elements, include microsatellites, terminal repeats, tandem repeats, minisatellites, satellite DNA, interspersed repeats, transposable elements (e.g., DNA transposons, retrotransposons (e.g., LTR-retrotransposons (HERVs) and LTR-retrotransposons (HERVs)), etc.), clustered regularly interspaced short palindromic repeats (CRISPR), direct repeats, inverted repeats, mirror repeats, and everted repeats. The genomic regions used in the methods described herein optionally include one or more repetitive elements. Additional details regarding repetitive elements are described in, for example, de Koning et al., “Repetitive elements may comprise over two-thirds of the human genome,” PLoS Genet 7.12 (2011), which is incorporated by reference.

Exon/intron or intron/exon junctions (splice junctions) typically include specific duplex sequence patterns in genomes and are involved in RNA splicing of mRNA. These sequences are optionally used as genomic regions in the methods described herein. Additional details regarding exon/intron or intron/exon junctions and related sequences are described in, for example, Mount, “A catalogue of splice junction sequences,” Nucleic Acids Research, 10(2):459-472 (1982), which is incorporated by reference.

A transcription start site (TSS) is the location where the first DNA nucleotide at the 5′-end of a given gene is transcribed into RNA. TSS sequences are optionally used as genomic regions in the methods described herein. Additional details regarding TSSs are described in, for example, Farman et al., “Nucleosomes positioning around transcriptional start site of tumor suppressor (Rbl2/p130) gene in breast cancer,” Molecular Biology Reports, 45(2):185-194 (2018), which is incorporated by reference.

In some embodiments, the methods, and related system and computer readable media implementations, disclosed herein include determining the cellular origin of DNA molecules from cfDNA samples using properties of those DNA molecules, such as epigenetic patterns exhibited by those molecules or fragments. As described herein, epigenetic changes in genomic sections are often accompanied by changes in chromatin organization and nucleosome positioning within those genomic sections. Accordingly, the methods and related aspects of this disclosure combine these sources of signal to increase the ability to detect the presence of targeted cells (e.g., diseased cells, such as tumor cells or the like), fetal cells, transplant donor cells, and the like) in cfDNA samples.

Any epigenetic site or locus that exhibits differential modifications (e.g., a post-replication modification or the like) between at least two cell or tissue types can be used to perform the methods and related aspects of the present disclosure. Examples of such sites, include methylation sites, acetylation sites, ubiquitylation sites, phosphorylation sites, sumoylation sites, ribosylation sites, citrullination sites, histone post-translational modification sites, histone variant sites, and/or the like. Examples of post-replication modifications, include 5-methyl-cytosine, 5-hydroxymethyl-cytosine, 5-carboxyl-cytosine, and 5-formyl-cytosine, among many others. Additional details regarding epigenetic sites or loci are described in, for example, Jin et al., “DNA Methylation: Superior or Subordinate in the Epigenetic Hierarchy?,” Genes Cancer, 2(6):607-617 (2011), Javaid et al., “Acetylation-and Methylation-Related Epigenetic Proteins in the Context of Their Target,” Genes (Basel), 8(8):196 (2017), Cao et al., “Histone Ubiquitination and Deubiquitination in Transcription, DNA Damage Response, and Cancer,” Front Oncol, 2:26 (2012), Rossetto et al., “Histone phosphorylation: A chromatin modification involved in diverse nuclear event,” Epigenetics, 7(10):1098-1108 (2012), Vranych et al., “SUMOylation and deimination of proteins: two epigenetic modifications involved in Giardia encystation,” Biochim Biophys Acta, 1843(9):1805-17 (2014), Sadakierska-Chudy et al., “A Comprehensive View of the Epigenetic Landscape. Part H: Histone Post-translational Modification, Nucleosome Level, and Chromatin Regulation by ncRNAs,” Neurotox Res, 27:172-197 (2015), Fuhrmann et al., “Protein Arginine Methylation and Citrullination in Epigenetic Regulation,” ACS Chem Biol, 11(3):654-668 (2016), Fan et al., “Metabolic regulation of histone post-translational modifications,” ACS Chem Biol, 10(1):95-108 (2015), and Henikoff et al., “Histone Variants and Epigenetics,” Cold Spring Harb Perspect Biol, 7(1) (2015), which are each incorporated by reference.

Epigenetic information can be obtained from cfDNA fragments using any technique known to those of ordinary skill in the art. In some embodiments, for example, DNA molecules from a given cfDNA sample are physically fractionated (e.g., fractionating with methyl-binding domain protein (“MBD”)-beads to stratify the cfDNA fragments into various degrees of methylation or the like) to generate partitions. In these embodiments, differential molecular tags and NGS-enabling adapters are applied to each of the two or more partitions to generate molecular tagged partitions. In addition, these embodiments also include assaying the molecular tagged partitions on an NGS instrument to generate sequence data for deconvoluting the sample into molecules that were differentially partitioned to generate the epigenetic information. In some embodiments, bisulfite sequencing techniques are also used to generate epigenetic information from cfDNA samples. Additional details regarding the analysis of epigenetic modifications that are optionally adapted for use in performing the methods disclosed herein are described in, for example, WO 2018/119452, filed Dec. 22, 2017, which is incorporated by reference.

In some embodiments, the methods, and related system and computer readable media implementations, disclosed herein include determining the cellular origin of DNA molecules from nucleic acid samples, for example, cfDNA samples, using properties of the sequences (e.g., sequence fragments/reads) that are ascertained via a sequencing process, such as fragmentomic patterns exhibited by those molecules or fragments. Human plasma DNA comprises a mixture of DNA fragments of different sizes, accordingly size of sequence fragments may form part of a fragmentomic signature. The modal size is approximately 166 base pairs (bp) and may be related to nucleosomal structure. Cell-free tumor-derived DNA in plasma of cancer patients has shorter modal sizes of approximately 143 bp. The size profiles of ctDNA may have a shorter median length and may be more variable in subjects with cancer than in subjects without cancer. Additionally, a pattern of cell-free DNA size peaks may be used to distinguish between tumor and non-tumor sequence fragments.

Cell-free tumor-derived DNA may exhibit different ends when compared to cell-free non-tumor-derived DNA, accordingly end motifs may form part of a fragmentomic signature. The ending sequences reveal overrepresentation of certain motifs that could be characterized by a range of nucleotides, such as 2-nucleotide oligomer (2-mer) or 4-mer motifs. Many human cancers exhibit down-regulation of the expression of DNASE1L3 which results in a reduced plasma DNA with DNASE1L3-associated end motifs. Plasma DNA end motifs demonstrate an advantage in that their maximal diagnostic power may be achieved with a relatively small number of DNA molecules analyzed. For example, on the basis of computer simulation, at a tumor DNA fraction of 10%, it would only require 50,000 plasma DNA molecules (DNA content of each cell is fragmented into about 20 million cell-free DNA molecules) to differentiate patients with and without hepatocellular carcinoma, whereas at least 7.5 million DNA molecules would be needed to detect a 1-megabase (Mb) copy number aberration. The detection of tumor-derived single-nucleotide variants in plasma DNA has been shown to need much higher sequencing depth (for example, >200 times haploid human genome coverage).

Double-stranded cell-free DNA may have blunt ends or jagged ends, accordingly presence and/or extent of a jagged end may form part of a fragmentomic signature. Different nucleases have different preferences for the generation of cleaved double-stranded DNA with blunt versus protruding or jagged ends. Jagged ends may be repaired with either methylated or unmethylated cytosines, and then the abundance of jagged ends may be measured by a change in methylation level from that of the genome. The frequencies of jagged ends have been found to be increased in ctDNA in cancer patients. The frequencies of jagged ends may be related to the relative activities between DNASE1 and DNASE1L3, with the former increasing and the latter decreasing the frequencies of jagged ends.

Plasma DNA fragmentation is a nonrandom process in which certain genomic regions are more prone to be cleaved and to be found at an end of a plasma DNA fragment, called “preferred end sites,” accordingly such sites may form part of a fragmentomic signature. These sites may differ for DNA molecules with different tissue sources. When cell-free DNA is aligned to the human genome, their ends tend to cluster at genomic locations (preferred end sites), which can be variable between DNA molecules that originate from different tissues. A window protection score, which may be calculated as the number of complete fragments minus the number of fragment endpoints within a given window size, may convey information about DNA protection from digestion, which can be used to infer nucleosome positioning. The genomic coverage and directional information of the cell-free DNA ending locations-namely upstream end or downstream end—are reflective of the chromatin structure of the tissue of origin (e.g., TF, transcription factor).

The predominant local positions of nucleosomes across the human genome in tissue(s) contributing to cfDNA may be inferred by comparing the distribution of aligned fragment endpoints, or a mathematical transformation thereof, to one or more reference maps. An example of values that can be used for fragmentomic analysis is a Windowed Protection Score (“WPS”) as described in PCT application WO2016/015058, which was developed to reflect such positioning, accordingly a WPS may form part of a fragmentomic signature. Specifically, it is expected that cfDNA fragment endpoints should cluster adjacent to nucleosome boundaries, while also being depleted on the nucleosome itself. The value of the WPS correlates with the locations of nucleosomes within strongly positioned arrays, as mapped by other groups with in vitro methods or ancient DNA. At other sites, the WPS correlates with genomic features such as DNase I hypersensitive (DHS) sites (e.g., consistent with the repositioning of nucleosomes flanking a distal regulatory element). Fragmentomic analysis typically involves determining a value (or values) based on the number of fragment endpoints that map to a specific genomic location (one base or more) as normalized for the amount of sequence data at or near the genomic location so as to fragmentomic values that can be input into models for comparing healthy and afflicted individuals in order determine the possible presence or absence of disease in the test subject. For example, if 10000 paired end reads have an end that map within 500 bp genomic region and 100 ends map to a single base location within that 500 bp region, then a value of 100/1000 could be a fragmentomic value for that single base locations. While not being bound by theory, fragmentomic values appear to be indicative of the presence or absence of proteins, e.g. histones or transcription factors, bound to the interrogated genomic regions. The presence or absence or such bound proteins is believed to affect the accessibility of nuclease to the DNA protected by the bound proteins.

In an embodiment, in a feature engineering step 104, input features for a machine learning step may be created by, for example, analyzing the sequence data, the epigenetic data, the fragmentomic data, combinations thereof, and the like. Additional or other data types may optionally be used for the feature engineering step. The method 100 may also comprise one or more transformation and/or clean-up processes at a data normalization step 106, such as, clean-up for sample prevalences (e.g., adjust for samples with a low number of a given nucleic acid variant, low number of samples, etc.), perform log transformations (e.g., Log (x+1) or Np·log 1p), and perform normalization (e.g., Yeo-Johnson normalization, min-max normalization, z-score normalization, and/or the like) (step 108).

The method 100 may comprise a machine learning step 108 that generates a machine learning model (e.g., classifier) according to a training dataset generated from the data obtained at step 102 (e.g., through creation of a training data set) and the input features from step 104. The machine learning model may be configured provide classify, predict, or otherwise determine one or more probabilities that the origin of a given nucleic acid variant present in a test sample is tumor or non-tumor. The machine learning step 108 may use any machine learning technique, for example, logistic regression or a deep learning technique. Exemplary models that can be used for training and classification, may include without limitations, one or more of: logistic regression, probit regression, decision trees, random forests, gradient boosting, support vector machines, k-nearest neighbors, neural networks, or an ensemble of more than one of these methods. Ensemble methods are meta-algorithms that combine several machine learning techniques into one predictive model in order to decrease variance (bagging), bias (boosting), or improve predictions (stacking). Most ensemble methods use a single base learning algorithm to produce homogeneous base learners, that is, learners of the same type, leading to homogeneous ensembles. There are also some methods that use heterogeneous learners, that is, learners of different types, leading to heterogeneous ensembles. In order for ensemble methods to be more accurate than any of its individual members, the base learners have to be as accurate as possible and as diverse as possible.

The method 100 may, at step 110, output a machine learning model/classifier that is configured to classify or otherwise predict the origin of a variant when provided with sequence data, epigenetic data, and or fragmentomic data associated with the variant.

The machine learning model/classifier may be used to determine an origin of a newly presented sequence fragment and/or variant in a test sample. The origin may be tumor derived or may be non-tumor derived. A sequence fragment and/or variant classified as tumor derived by the machine learning model/classifier may be used to direct treatment of a subject. It may have been previously unknown whether the subject has a disease or it may be known that the subject has a disease. The disease may be cancer. The methods may comprise administering one or more therapies to the subject to treat the disease. The therapies may comprise administering chemotherapy, administering radiation therapy, or performing surgery to resect all or a portion of the tumor. The methods may comprise assisting in a communication of determination of the origin as being tumor derived to a subject associated with the test sample.

### II. Exemplary Systems and Methods

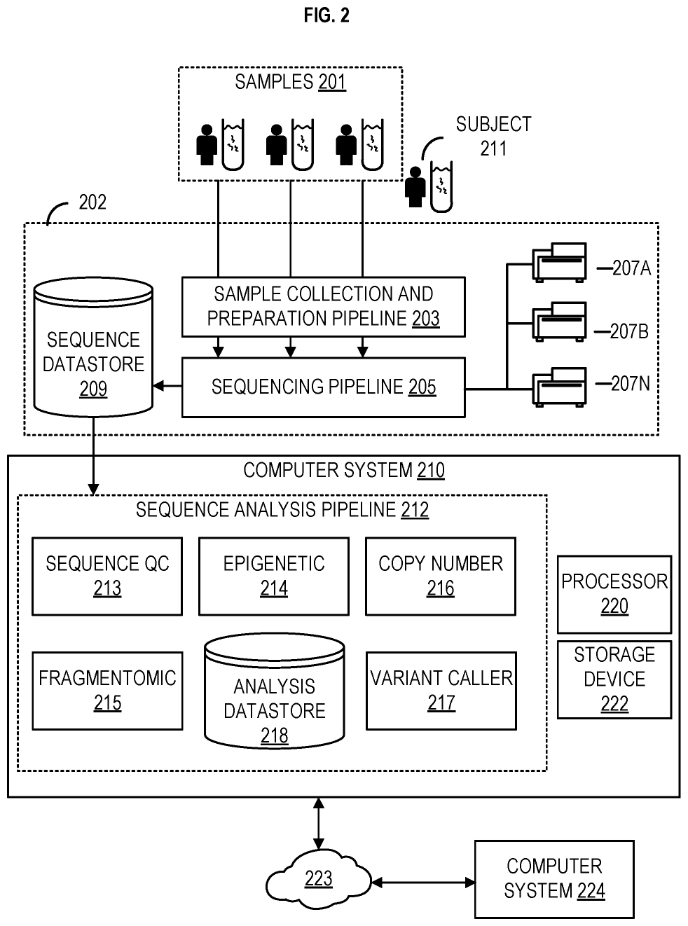

FIG. 2 illustrates an example of a system 200 for determining an origin of a variant of a test subject 211, according to an embodiment of the present disclosure. The system 200 may process one or more samples 201 from the subject 211 to generate sequence reads for variant detection and variant origin determination. The system 200 may include a laboratory system 202, a computer system 210, and/or other components. It should be noted that the laboratory system 202 and the computer system 210 may be remote from one another, and connected to one another through a computer network (not illustrated). The laboratory system 202 may include a sample collection and preparation pipeline 203, a sequencing pipeline 205, a sequence read datastore 209, and/or other components. The sequencing pipeline 205 may include one or more sequencing devices 207 (illustrated in FIG. 2 as sequencing devices 207a . . . n).

The methods of this disclosure may have a wide variety of uses in the manipulation, preparation, identification, quantification, and/or analysis of cell-free nucleic acids. As shown in FIG. 2, the sample collection and preparation pipeline 203 may include obtaining cfDNA reference samples 201 from one or more reference subjects and a cfDNA test sample 211 from a test subject. As described herein, a polynucleotide can comprise any type of nucleic acid, such as DNA and/or RNA. For example, if a polynucleotide is DNA, it can be genomic DNA, complementary DNA (cDNA), or any other deoxyribonucleic acid. A polynucleotide can also be a cell-free nucleic acid such as cell-free DNA (cfDNA). For example, the polynucleotide can be circulating cfDNA. Circulating cfDNA may comprise DNA shed from bodily cells via apoptosis or necrosis. cfDNA shed via apoptosis or necrosis may originate from normal (e.g., healthy) bodily cells. Where there is abnormal tissue growth, such as for cancer, tumor DNA may be shed. The circulating cfDNA can comprise circulating tumor DNA (ctDNA).

a. Samples

Isolation and extraction of cell free polynucleotides may be performed through collection of samples using a variety of techniques. A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can comprise various amounts of nucleic acid. Typically, the amount of nucleic acid in a given sample is equated with multiple genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations). In some embodiments of the present disclosure, cell free nucleic acids in a subject may derive from a tumor. For example cell-free DNA isolated from a subject can comprise ctDNA.

Exemplary amounts of cell-free nucleic acids in a sample before amplification typically range from about 1 femtogram (fg) to about 1 microgram (g), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

b. Partitioning; Analysis of Epigenetic Characteristics

In certain embodiments described herein, a population of different forms of nucleic acids (e.g., hypermethylated and hypomethylated DNA in a sample from the subject, such as tagged DNA or an aliquot thereof) can be physically partitioned based on one or more characteristics of the nucleic acids prior to analysis, e.g., sequencing, or tagging and sequencing. This approach can be used to determine, for example, whether hypermethylation variable epigenetic target regions show hypermethylation characteristic of tumor cells or hypomethylation variable epigenetic target regions show hypomethylation characteristic of tumor cells or otherwise indicative of the presence of disease. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some embodiments, the partitions are differentially tagged and then recombined before dividing the sample into first and second aliquots, followed by subsequent steps of methods described herein. In some embodiments, the sample that is divided into the first and second aliquots is a partition, such as a hypomethylated partition, and the second aliquot is combined with at least one other partition, such as a hypermethylated partition, before undergoing enrichment and/or other steps of the method.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively, or additionally, a heterogeneous population of nucleic acids can be partitioned into nucleic acid molecules associated with nucleosomes and nucleic acid molecules devoid of nucleosomes. Alternatively, or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced.

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine.

In some embodiments, the nucleic acids in the original population can be single-stranded and/or double-stranded. Partitioning based on single v. double stranded-ness of the nucleic acids can be accomplished by, e.g. using labelled capture probes to partition ssDNA and using double stranded adapters to partition dsDNA.

The affinity agents can be antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target.

Examples of capture moieties contemplated herein include methyl binding domain (MBDs) and methyl binding proteins (MBPs) as described herein.

Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4 (RbAp48) and SANT domain peptides.

Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (Thermo Fisher Scientific). Subsequently, additional partitioning may involve eluting fragments having different levels of methylation by adjusting the salt concentration in a solution with the methyl-binding domain and bound fragments. As salt concentration increases, fragments having greater methylation levels are eluted.

In some instances, the final partitions are representatives of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (Thermo Fisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can repeat themselves to create various partitions such as a hypomethylated partition (e.g., representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 mM to about 2500 mM NaCl. In one embodiment, the process results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

In some embodiments, e.g., wherein an epigenetic target region set is captured, sample DNA (for e.g., between 1 and 300 ng) is mixed with an appropriate amount of methyl binding domain (MBD) buffer (the amount of MBD buffer depends on the amount of DNA used) and magnetic beads conjugated with MBD proteins and incubated overnight. Methylated DNA (hypermethylated DNA) binds the MBD protein on the magnetic beads during this incubation. Non-methylated (hypomethylated DNA) or less methylated DNA (intermediately methylated) is washed away from the beads with buffers containing increasing concentrations of salt. For example, one, two, or more fractions containing non-methylated, hypomethylated, and/or intermediately methylated DNA may be obtained from such washes. Finally, a high salt buffer is used to elute the heavily methylated DNA (hypermethylated DNA) from the MBD protein. In some embodiments, these washes result in three partitions (hypomethylated partition, intermediately methylated fraction and hypermethylated partition) of DNA having increasing levels of methylation.

In some embodiments, the three partitions of DNA are desalted and concentrated in preparation for the enzymatic steps of library preparation.

In some embodiments, the methylation signature of molecules can be determined by methods such as MeDIP-seq, MBD-seq, BS-seq, Ox-BS-seq, TAP-seq, ACE-seq, hmC-seal, and TAB-seq. See, e.g., Schutsky, E. K. et al. Nondestructive, base-resolution sequencing of 5-hydroxymethylcytosine using a DNA deaminase. Nature Biotech, 2018; doi.10.1038/nbt.4204 (ACE-Seq); Yu, Miao et al. Base-resolution analysis of 5-hydroxymethylcytosine in the Mammalian Genome. Cell, 2012; 149(6):1368-80 (TAB-Seq); Han, D. A highly sensitive and robust method for genome-wide 5hmC profiling of rare cell populations. Mol Cell. 2016; 63(4):711-719 (5hmC-Seal); Shen, S. Y. et al. Sensitive tumour detection and classification using plasma cell-free DNA methylomes. Nature. 2018; 563(7732):579-583 (cfMeDIP); Nair, S S et al. Comparison of methyl-DNA immunoprecipitation (MeDIP) and methyl-CpG binding domain (MBD) protein capture for genome-wide DNA. Epigenetics. 2011; 6(1):34-44. In some embodiments, the methylation signature of molecules can be determined by treating the sample with one or more methylation sensitive restriction enzymes (MSRE) and/or methylation dependent restriction enzymes (MDRE). In some embodiments, any of the above methods can be used either alone or in combination, to determine the methylation signature of the molecules.

c. Nucleic Acid Tags

In some embodiments, the nucleic acid molecules (from the sample of polynucleotides) may be tagged with sample indexes and/or molecular barcodes (referred to generally as “tags”). Tags may be incorporated into or otherwise joined to adapters by chemical synthesis, ligation (e.g., blunt-end ligation or sticky-end ligation), or overlap extension polymerase chain reaction (PCR), among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing steps are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps. In some embodiments, the sample indexes are introduced after sequence capturing steps are performed. In some embodiments, molecular barcodes are incorporated to the nucleic acid molecules (e.g. cfDNA molecules) in a sample through adapters via ligation (e.g., blunt-end ligation or sticky-end ligation). In some embodiments, sample indexes are incorporated to the nucleic acid molecules (e.g. cfDNA molecules) in a sample through overlap extension polymerase chain reaction (PCR). Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original nucleic acid molecule in the sample, start and stop genomic positions corresponding to the sequence of the original nucleic acid molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original nucleic acid molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

In certain embodiments described herein, a population of different forms of nucleic acids (e.g., hypermethylated and hypomethylated DNA in a sample) can be physically partitioned prior to analysis, e.g., sequencing, or tagging and sequencing. This approach can be used to determine, for example, whether hypermethylation variable epigenetic target regions show hypermethylation characteristic of tumor cells or hypomethylation variable epigenetic target regions show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged—i.e., each partition can have a different set of molecular barcodes. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively, or additionally, a heterogeneous population of nucleic acids can be partitioned into nucleic acid molecules associated with nucleosomes and nucleic acid molecules devoid of nucleosomes. Alternatively, or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially tagged with molecular barcodes, and the partitions are pooled together prior to sequencing. In other instances, the different forms are separately sequenced. In some embodiments, a single tag can be used to label a specific partition. In some embodiments, multiple different tags can be used to label a specific partition. In embodiments employing multiple different tags to label a specific partition, the set of tags used to label one partition can be readily differentiated from the set of tags used to label other partitions. In some embodiments, a tag can be multifunctional—i.e., it can simultaneously act as a molecular identifier (i.e., molecular barcode), partition identifier (i.e., partition tag) and sample identifier (i.e., sample index). For example, if there are four DNA samples and each DNA sample is partitioned into three partitions, then the DNA molecules in each of the twelve partitions (i.e., twelve partitions for the four DNA samples in total) can be tagged with a separate set of tags such that the tag sequence attached to the DNA molecule reveals the identity of the DNA molecule, the partition it belongs to and the sample from which it was originated. In some embodiments, a tag can be used both as a molecular barcode and as a partition tag. For example, if a DNA sample is partitioned into three partitions, then DNA molecule in each partition is tagged with a separated set of tags such that the tag sequence attached to a DNA molecule reveals the identity of the DNA molecule and the partition it belongs to. In some embodiments, a tag can be used both as a molecular barcode and as a sample index. For example, if there are four DNA samples, then DNA molecules in each sample with be tagged with a separate set of tags that can be distinguishable from each sample such that the tag sequence attached to the DNA molecule serves as a molecule identifier and as a sample identifier.

In one embodiment, partition tagging comprises tagging molecules in each partition with a partition tag. After re-combining partitions and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more epigenetic characteristics, the final tagged molecules in the library may no longer possess that epigenetic characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique and/or non-unique.

One exemplary format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

After sequencing, analysis of reads to detect genetic variants can be performed on a partition-by-partition level, as well as a whole nucleic acid population level. Tags are used to sort reads from different partitions. Analysis can include in silico analysis to determine genetic and epigenetic variation (one or more of methylation, chromatin structure, etc.) using sequence information, genomic coordinates length, coverage and/or copy number. In some embodiments, higher coverage can correlate with higher nucleosome occupancy in genomic region while lower coverage can correlate with lower nucleosome occupancy or a nucleosome depleted region (NDR).

d. Nucleic Acid Amplification

Sample nucleic acids flanked by adapters are typically amplified by PCR and other amplification methods using nucleic acid primers binding to primer binding sites in adapters flanking a DNA molecule to be amplified as part of the sample collection and preparation pipeline 203. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular tags and/or sample indexes/tags to a nucleic acid molecule using conventional nucleic acid amplification methods. The amplifications are typically conducted in one or more reaction mixtures. Molecular tags and sample indexes/tags are optionally introduced simultaneously, or in any sequential order. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

e. Nucleic Acid Enrichment

In some embodiments, sequences are enriched prior to sequencing the nucleic acids as part of the sample collection and preparation pipeline 203. Enrichment is optionally performed for specific target regions or nonspecifically (“target sequences”). In some embodiments, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some embodiments, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain embodiments, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

f. Nucleic Acid Sequencing

As shown in FIG. 2, after extraction and isolation of cfDNA from samples via the sample collection and preparation pipeline 203, the cfDNA may be sequenced via the sequencing pipeline 205 including one or more sequencing devices 207. Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain markers of cancer or of other diseases. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U). Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Additional details regarding nucleic acid sequencing, including the formats and applications described herein are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

i. Sequencing Panel

To improve the likelihood of detecting genomic regions of interest and optionally, tumor indicating mutations, the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced). A sequencing panel can target a plurality of different genes or regions, for example, to detect a single cancer, a set of cancers, or all cancers. Alternatively, DNA may be sequenced by whole genome sequencing (WGS) or other unbiased sequencing method without the use of a sequencing panel. Examples of suitable panel and targets for use in panels can be found in the epigenetic targets described in International Application WO2020160414, filed Jan. 31, 2020, which is incorporated by reference in its entirety.

In some aspects, a panel that targets a plurality of different genes or genomic regions (e.g., CHIP genes, transcriptional factor binding regions, distal regulatory elements (DREs), repetitive elements, intron-exon junctions, transcriptional start sites (TSSs), and/or the like) is selected such that a determined proportion of subjects having a cancer exhibits a genetic variant or tumor marker in one or more different genes in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Genes included in this panel may comprise one or more of: ATM, ATR, BAP1, BARD1, BRCA1, BRCA2, BRIP1, CDK12, CHEK1, CHEK2, FANCA, FANCL, HDAC2, MRE11, NBN, PALB2, RAD50, RAD51, RAD51B, RAD51C, RAD51D, RAD54L, XRCC2, XRCC3 DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some embodiments, markers for a tissue of origin are tissue-specific epigenetic markers.

Some examples of listings of genomic locations of interest may be found in Table 3 and Table 4. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or 97 of the genes of Table 3. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, or 70 of the SNVs of Table 3. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 3. In some embodiments, genomic locations used in the methods of the present disclosure comprise at least 1, at least 2, at least 3, at least 4, at least 5, or 6 of the fusions of Table 3. In some embodiments, genomic locations used in the methods of the present disclosure comprise at least a portion of at least 1, at least 2, or 3 of the indels of Table 3. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or 115 of the genes of Table 4. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 4. In some embodiments, genomic locations used in the methods of the present disclosure comprise at least 1, at least 2, at least 3, at least 4, at least 5, or 6 of the fusions of Table 4. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. Each of these genomic locations of interest may be identified as a backbone region or hot-spot region for a given bait set panel. An example of a listing of hot-spot genomic locations of interest may be found in Table 5. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5. Each hot-spot genomic location is listed with several characteristics, including the associated gene, chromosome on which it resides, the start and stop position of the genome representing the gene's locus, the length of the gene's locus in base pairs, the exons covered by the gene, and the critical feature (e.g., type of mutation) that a given genomic location of interest may seek to capture.

TABLE 3

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | CDKN2B | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | |
| RIT1 | ROS1 | SMAD4 | SMO | SRC | STK11 | | | | |
| TERT | TP53 | TSC1 | VHL | | | | | | |

TABLE 4

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | DDR2 | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | ATM |
| RIT1 | ROS1 | SMAD4 | SMO | MAPK1 | STK11 | | | | |
| TERT | TP53 | TSC1 | VHL | MAPK3 | MTOR | | | | |
| NTRK3 | | | | | | | | | APC |
| | | | | | | | | | ARID1A |
| | | | | | | | | | BRCA1 |
| | | | | | | | | | BRCA2 |
| | | | | | | | | | CDH1 |
| | | | | | | | | | CDKN2A |
| | | | | | | | | | GATA3 |
| | | | | | | | | | KIT |
| | | | | | | | | | MLH1 |
| | | | | | | | | | MTOR |
| | | | | | | | | | NF1 |
| | | | | | | | | | PDGFRA |
| | | | | | | | | | PTEN |
| | | | | | | | | | RB1 |
| | | | | | | | | | SMAD4 |
| | | | | | | | | | STK11 |
| | | | | | | | | | TP53 |
| | | | | | | | | | TSC1 |
| | | | | | | | | | VHL |

TABLE 5

| | Chrom- | Start | Stop | Length | Exons | |
|---|---|---|---|---|---|---|
| Gene | osome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

In some embodiments, the one or more regions in the panel comprise one or more loci from one or a plurality of genes for detecting residual cancer after surgery. This detection can be earlier than is possible for existing methods of cancer detection. In some embodiments, the one or more genomic locations in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some embodiments, the methods described herein detect cancer in high risk patients earlier than is possible for existing methods of cancer detection.

A genomic location may be selected for inclusion in a sequencing panel based on a number of subjects with a cancer that have a tumor marker in that gene or region. A genomic location may be selected for inclusion in a sequencing panel based on prevalence of subjects with a cancer and a tumor marker present in that gene. Presence of a tumor marker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A databased may comprise information about regulatory elements or genomic regions in tumor samples. The information relating to the sequenced tumor samples may include the frequency various genetic variants and describe the genes or regions in which the genetic variants occur. The genetic variants may be tumor markers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with tumor marker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or genomic section may be selected for a panel where the frequency of a tumor marker is significantly greater in sampled tumor tissue or circulating tumor DNA than found in a given background population. A combination of genomic locations may be selected for inclusion of a panel such that at least a majority of subjects having a cancer may have a tumor marker or genomic region present in at least one of the genomic location or genes in the panel. The combination of genomic location may be selected based on data indicating that, for a particular cancer or set of cancers, a majority of subjects have one or more tumor markers in one or more of the selected regions. For example, to detect cancer 1, a panel comprising regions A, B, C, and/or D may be selected based on data indicating that 90% of subjects with cancer 1 have a tumor marker in regions A, B, C, and/or D of the panel. Alternately, tumor markers may be shown to occur independently in two or more regions in subjects having a cancer such that, combined, a tumor marker in the two or more regions is present in a majority of a population of subjects having a cancer. For example, to detect cancer 2, a panel comprising regions X, Y, and Z may be selected based on data indicating that 90% of subjects have a tumor marker in one or more regions, and in 30% of such subjects a tumor marker is detected only in region X, while tumor markers are detected only in regions Y and/or Z for the remainder of the subjects for whom a tumor marker was detected. Tumor markers present in one or more genomic locations previously shown to be associated with one or more cancers may be indicative of or predictive of a subject having cancer if a tumor marker is detected in one or more of those regions 50% or more of the time. Computational approaches such as models employing conditional probabilities of detecting cancer given a cancer frequency for a set of tumor markers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, bisulfate sequencing, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the minor allele frequency for a given genetic variant. The minor allele frequency may refer to the frequency at which minor alleles (e.g., not the most common allele) occurs in a given population of nucleic acids, such as a sample. Genetic variants at a low minor allele frequency may have a relatively low frequency of presence in a sample. In some cases, the panel allows for detection of genetic variants at a minor allele frequency of at least 0.0001%, 0.001%, 0.005%, 0.01%, 0.05%, 0.1%, or 0.5%. The panel can allow for detection of genetic variants at a minor allele frequency of 0.001% or greater. The panel can allow for detection of genetic variants at a minor allele frequency of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of tumor markers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of tumor markers at a frequency in a sample as low as 1.0%. The panel can allow for detection of tumor markers at a frequency in a sample as low as 0.75%. The panel can allow for detection of tumor markers at a frequency in a sample as low as 0.5%. The panel can allow for detection of tumor markers at a frequency in a sample as low as 0.25%. The panel can allow for detection of tumor markers at a frequency in a sample as low as 0.1%. The panel can allow for detection of tumor markers at a frequency in a sample as low as 0.075%. The panel can allow for detection of tumor markers at a frequency in a sample as low as 0.05%. The panel can allow for detection of tumor markers at a frequency in a sample as low as 0.025%. The panel can allow for detection of tumor markers at a frequency in a sample as low as 0.01%. The panel can allow for detection of tumor markers at a frequency in a sample as low as 0.005%. The panel can allow for detection of tumor markers at a frequency in a sample as low as 0.001%. The panel can allow for detection of tumor markers at a frequency in a sample as low as 0.0001%. The panel can allow for detection of tumor markers in sequenced cfDNA at a frequency in a sample as low as 1.0% to 0.0001%. The panel can allow for detection of tumor markers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the genomic positions in the panel.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The locations comprising genomic regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected. In some embodiments, a genomic region of the panel may comprise one or more of the following genes: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET.

The regions in the panel can be selected so that they comprise sequences differentially transcribed across one or more tissues. In some cases, the locations comprising genomic regions can comprise sequences transcribed in certain tissues at a higher level compared to other tissues. For example, the locations comprising genomic regions can comprise sequences transcribed in certain tissues but not in other tissues.

The genomic locations in the panel can comprise coding and/or non-coding sequences. For example, the genomic locations in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the genomic locations in the panel can comprise sequences in non-coding RNA, e.g., ribosomal RNA, transfer RNA, Piwi-interacting RNA, and microRNA.

The genomic locations in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the cancer with a sensitivity of 100%.

The genomic locations in the panel can be selected to detect (diagnose) a cancer with a desired level of specificity (e.g., through the detection of one or more genetic variants). For example, the genomic locations in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the one or more genetic variant with a specificity of 100%.

The genomic locations in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, genomic locations in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The genomic locations in the panel can be selected to detect (diagnose) a cancer with a desired accuracy. As used herein, the term “accuracy” may refer to the ability of a test to discriminate between a disease condition (e.g., cancer) and healthy condition. Accuracy may be can be quantified using measures such as sensitivity and specificity, predictive values, likelihood ratios, the area under the ROC curve, Youden's index and/or diagnostic odds ratio.

Accuracy may presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The genomic locations in the panel can be selected to detect cancer with an accuracy of 100%.

A panel may be selected to be highly sensitive and detect low frequency genetic variants. For instance, a panel may be selected such that a genetic variant or tumor marker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a sensitivity of 70% or greater. A panel may be selected to detect a tumor marker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or tumor marker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a specificity of 70% or greater. A panel may be selected to detect a tumor marker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or tumor marker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with an accuracy of 70% or greater. A panel may be selected to detect a tumor marker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or tumor marker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

In an embodiment, utilizing the sequencing pipeline 205, the panel may be subjected to one or more of: whole-genome bisulfite sequencing (WGBS) interrogating genome-wide methylation patterns, whole-genome sequencing (WGS), and/or targeted sequencing approaches interrogating copy-number variants (CNVs) and single-nucleotide variants (SNVs).

Genetic and/or epigenetic information obtained from DNA of the subject can be combined to provide a determination of whether a subject has a cancer or a likelihood that the subject has a cancer. Detailed descriptions of how to analyze cell free human DNA for both genetic and epigenetic variants associated with cancer can be found in U.S. provisional patent application 62/799,637, which is herein incorporated by reference in its entirety. Additional guidance for analyzing cell free DNA for the detecting cancer can be found in, among other places U.S. Pat. No. 9,834,822, PCT application WO2018064629A1, and PCT application WO2017106768A1.

Various embodiments include the step of sequencing DNA (e.g., cfDNA) for the purpose of detecting genetic variants in genes associated with cancer. Various embodiments also include the step of sequencing DNA (e.g., cfDNA) for the purpose of detecting epigenetic variants in genes associated with cancer, for example, but not limited to, include DNA sequences that are differentially methylated in cancerous and noncancerous cells and nucleosomal fragmentation patterns such as those described in US published patent application US2017/0211143.

In some embodiments, a captured set of nucleic acid, e.g., comprising DNA (such as cfDNA) is provided. With respect to the disclosed methods, the captured set of DNA may be provided, e.g., following capturing, and/or separating steps as described herein. The captured set may comprise DNA corresponding to one or both of a sequence-variable target region set and an epigenetic target region set. In some embodiments, the captured set comprises DNA corresponding to a sequence-variable target region set, and an epigenetic target region set. In all embodiments described herein involving a sequence-variable target region set and an epigenetic target region set, the sequence-variable target region set comprises regions not present in the epigenetic target region set and vice versa, although in some instances a fraction of the regions may overlap (e.g., a fraction of genomic positions may be represented in both target region sets).

Methylation Target Region Set

In some embodiments, an epigenetic target region set is captured. The epigenetic target region set may comprise one or more types of target regions likely to differentiate DNA from neoplastic (e.g., tumor or cancer) cells and from healthy cells, e.g., non-neoplastic circulating cells. The epigenetic target region set can be analyzed in various ways, including methods that do not depend on a high degree of accuracy in sequence determination of specific nucleotides within a target. Exemplary types of such regions are discussed in detail herein. In some embodiments, methods according to the disclosure comprise determining whether cfDNA molecules corresponding to the epigenetic target region set comprise or indicate cancer-associated epigenetic modifications (e.g., hypermethylation in one or more hypermethylation variable target regions; one or more perturbations of CTCF binding; and/or one or more perturbations of transcription start sites) and/or copy number variations (e.g., focal amplifications). Such analyses can be conducted by sequencing and require less data (e.g., number of sequence reads or depth of sequencing coverage) than determining the presence or absence of a sequence mutation such as a base substitution, insertion, or deletion. The epigenetic target region set may also comprise one or more control regions, e.g., as described herein.

In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

Hypermethylation Variable Target Regions

In some embodiments, the epigenetic target region set comprises one or more hypermethylation variable target regions. In general, hypermethylation variable target regions refer to regions where an increase in the level of observed methylation indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation variable target regions comprising the genes or portions thereof based on the colorectal cancer (CRC) studies is provided in Table 6. Many of these genes likely have relevance to cancers beyond colorectal cancer; for example, TP53 is widely recognized as a critically important tumor suppressor and hypermethylation-based inactivation of this gene may be a common oncogenic mechanism.

TABLE 6
Exemplary hypermethylation target regions (genes or
portions thereof) based on CRC studies.

| | Additional | |
|---|---|---|
| Gene Name | Gene Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | MGMT | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, the hypermethylation variable target regions comprise a plurality of genes or portions thereof listed in Table 6, e.g., at least 10%, 20%, 30%, 40%, 50%, 60%, 70%, 80%, 90%, or 100% of the genes or portions thereof listed in Table 6. For example, for each locus included as a target region, there may be one or more probes with a hybridization site that binds between the transcription start site and the stop codon (the last stop codon for genes that are alternatively spliced) of the gene. In some embodiments, the one or more probes bind within 300 bp upstream and/or downstream of the genes or portions thereof listed in Table 6, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5(5):492-504 (2016); Skvortsova et al., Br. J. Cancer. 94(10):1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation variable target regions comprising genes or portions thereof based on the lung cancer studies is provided in Table 7. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 6 and 7, indicating generality.

TABLE 7
Exemplary hypermethylation target regions (genes
or portions thereof) based on lung cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 2 may be combined with any of the embodiments described above concerning target regions identified in Table 1. In some embodiments, the hypermethylation variable target regions comprise a plurality of genes or portions thereof listed in Table 1 or Table 2, e.g., at least 10%, 20%, 30%, 40%, 50%, 60%, 70%, 80%, 90%, or 100% of the genes or portions thereof listed in Table 1 or Table 2.

Additional hypermethylation target regions may be obtained, e.g., from the Cancer Genome Atlas. Kang et al., Genome Biology 18:53 (2017), describe construction of a probabilistic method called Cancer Locator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

Hypomethylation Variable Target Regions

Global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions, where a decrease in the level of observed methylation indicates an increased likelihood that a sample (e.g., of cfDNA) contains DNA produced by neoplastic cells, such as tumor or cancer cells.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1, e.g., according to the hg19 or hg38 human genome construct. In some embodiments, the hypomethylation variable target regions overlap or comprise one or both of these regions.

CTCF Binding Regions

CTCF is a DNA-binding protein that contributes to chromatin organization and often colocalizes with cohesin. Perturbation of CTCF binding sites has been reported in a variety of different cancers. See, e.g., Katainen et al., Nature Genetics, doi:10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. For example, details regarding sequencing-based fragment length analysis are provided in Snyder et al., Cell 164:57-68 (2016); WO 2018/009723; and US20170211143A1, each of which are incorporated herein by reference.

Thus, perturbations of CTCF binding result in variation in the fragmentation patterns of cfDNA. As such, CTCF binding sites represent a type of fragmentation variable target regions.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13, e.g., according to the hg19 or hg38 human genome construct.

Accordingly, in some embodiments, the epigenetic target region set includes CTCF binding regions. In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above.

In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and/or downstream regions of the CTCF binding sites.

Transcription Start Sites

Transcription start sites may also show perturbations in neoplastic cells. For example, nucleosome organization at various transcription start sites in healthy cells of the hematopoietic lineage—which contributes substantially to cfDNA in healthy individuals—may differ from nucleosome organization at those transcription start sites in neoplastic cells. This results in different cfDNA patterns that can be detected by sequencing, for example, as discussed generally in Snyder et al., Cell 164:57-68 (2016); WO 2018/009723; and US20170211143A1.

Thus, perturbations of transcription start sites also result in variation in the fragmentation patterns of cfDNA. As such, transcription start sites also represent a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34(Database issue): D86-D89 (2006), which is incorporated herein by reference.

Accordingly, in some embodiments, the epigenetic target region set includes transcriptional start sites. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and/or downstream regions of the transcription start sites.

Methylation Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes control hypomethylated regions that are expected to be hypomethylated in essentially all samples. In some embodiments, the epigenetic target region set includes control hypermethylated regions that are expected to be hypermethylated in essentially all samples.

Copy Number Variations; Focal Amplifications

Although copy number variations such as focal amplifications are somatic mutations, they can be detected by sequencing based on read frequency in a manner analogous to approaches for detecting certain epigenetic changes such as changes in methylation. As such, regions that may show copy number variations such as focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

g. Sequence Analysis Pipeline

In an embodiment, after sequencing, sequence reads and any associated data may be stored in the sequence datastore 209. The sequence reads can be stored in any format. The sequence datastore 209 may be local and/or remote to a location where sequencing is performed. As shown in FIG. 2, the stored reads may be subjected to a sequence analysis pipeline 212.

i. Sequence Quality Control

The sequence analysis pipeline 212 may include a sequence quality control (QC) component 213 that may filter sequence fragments/reads from the laboratory system 102. The sequence QC component 213 may assign a quality score to one or more sequence fragments/reads. A quality score may be a representation of sequence fragments/reads that indicates whether those sequence fragments/reads may be useful in subsequent analysis based on a threshold. In some cases, some sequence fragments/reads are not of sufficient quality or length to perform a subsequent mapping step. Sequence fragments/reads with a quality score at least 90%, 95%, 99%, 99.9%, 99.99% or 99.999% may be filtered out of a data set of sequence fragments/reads. In other cases, sequence fragments/reads assigned a quality scored at least 90%, 95%, 99%, 99.9%, 99.99% or 99.999% may be filtered out of the data set.

Sequence fragments/reads that meet a specified quality score threshold may be mapped to a reference genome by the sequence QC component 213. After mapping alignment, sequence fragments/reads may be assigned a mapping score. A mapping score may be a representation of sequence fragments/reads mapped back to the reference sequence indicating whether each position is or is not uniquely mappable. Sequence fragments/reads with a mapping score at least 90%, 95%, 99%, 99.9%, 99.99% or 99.999% may be filtered out of the data set. In other cases, sequencing fragments/reads assigned a mapping scored less than 90%, 95%, 99%, 99.9%, 99.99% or 99.999% may be filtered out of the data set.

ii. Epigenetic Component

In an embodiment, an epigenetic component 214 may analyze sequence fragments/reads to determine epigenetic data. Epigenetic data may include, for example, information regarding DNA methylation, histone states or modifications, inflammation-mediated cytosine damage products, protein binding, or other molecular states reflected in the nucleic acid fragment analyzed that are not ascertained solely from the nucleotide base sequence, e.g., the methylation status of give base or set bases. The epigenetic data may be used as an epigenetic signature. Epigenetic data may be determined by any means known in the art. The epigenetic data may be stored in the analysis datastore 218.

In accordance with the present description, cfDNA fragments from a sample 201 and/or a subject 211 may be treated in the sample collection and preparation pipeline 203, for example by converting unmethylated cytosines to uracils, sequenced according to the sequencing pipeline 205 and the sequence fragments/reads may be compared by the epigenetic component 214 to a reference genome to identify the methylation states at specific CpG sites within the sequence fragments/reads. Each CpG site may be methylated or unmethylated. Identification of anomalously methylated fragments, in comparison to healthy individuals, may provide insight into a subject's cancer status. DNA methylation anomalies (compared to healthy controls) can cause different effects, which may contribute to cancer. Methylation typically occurs in deoxyribonucleic acid (DNA) when a hydrogen atom on the pyrimidine ring of a cytosine base is converted to a methyl group, forming 5-methylcytosine. In particular, methylation tends to occur at dinucleotides of cytosine and guanine referred to herein as “CpG sites.” Anomalous DNA methylation can be identified as hypermethylation or hypomethylation, both of which may be indicative of cancer status. Throughout this disclosure, hypermethylation and hypomethylation may be characterized for a sequence fragment/read, if the sequence fragment/read comprises more than a threshold number of CpG sites with more than a threshold percentage of those CpG sites being methylated or unmethylated. Example thresholds for numbers of CpG sites include more than 3, 4, 5, 6, 7, 8, 9, 10, etc. Example percentage thresholds of methylation or unmethylation include more than 80%, 85%, 90%, or 95%, or any other percentage within the range of 50%-100%. Those of skill in the art will appreciate that the principles described herein are equally applicable for the detection of methylation in a non-CpG context, including non-cytosine methylation.

In an embodiment, the epigenetic component 214 may be configured to determine a location and methylation state for each CpG site based on alignment to a reference genome. The epigenetic component 214 may generate a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate. Indeterminate methylation states may originate from sequencing errors and/or disagreements between methylation states of a DNA fragment's complementary strands. The methylation state vectors may be stored in the analysis datastore 218 for later use and processing. Further, the epigenetic component 214 may remove duplicate reads or duplicate methylation state vectors from a single sample. The epigenetic component 214 may determine that a certain fragment with one or more CpG sites has an indeterminate methylation status over a threshold number or percentage and may exclude such fragments.

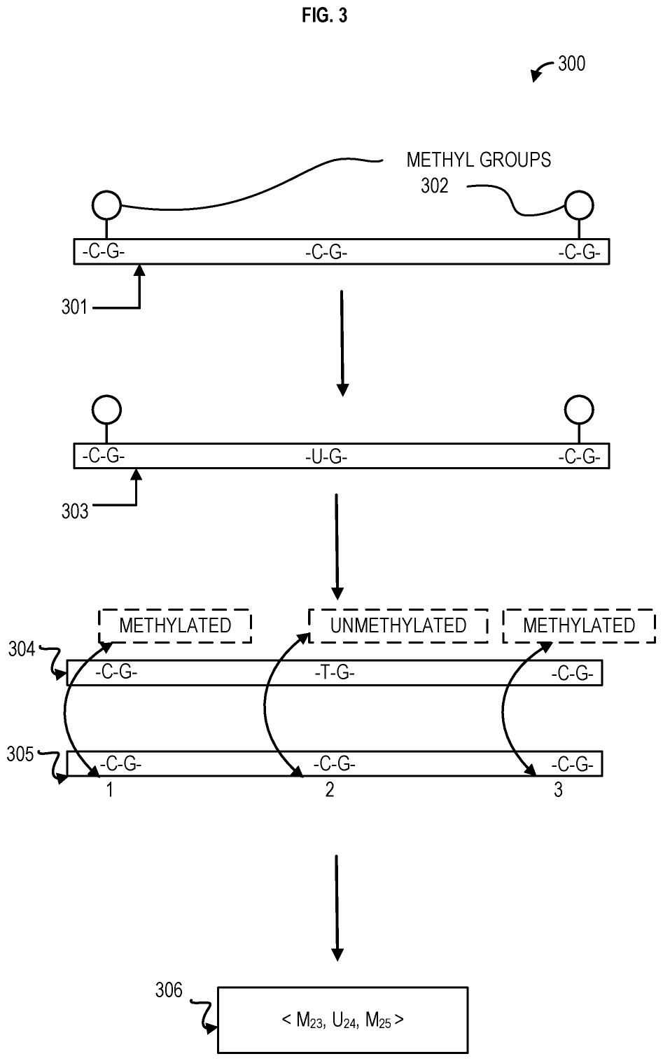

FIG. 3 is an illustration of a method 300 for sequencing a cfDNA molecule to obtain a methylation state vector. As an example, the laboratory system 202 receives a cfDNA molecule 301 that, in this example, contains three CpG sites. As shown, the first and third CpG sites of the cfDNA molecule 301 are methylated 302. As part of the sample collection and preparation pipeline 203, the cfDNA molecule 301 is converted to generate a converted cfDNA molecule 303. The second CpG site which was unmethylated has its cytosine converted to uracil but the first and third CpG sites were not converted.

After conversion, the sequencing pipeline 205 is used to generating sequence fragments/reads 304. The epigenetic component 214 may be configured to align the sequence fragment/read 304 to a reference genome 305. The reference genome 305 provides context as to what position in a human genome the fragment cfDNA originates. In this simplified example, the epigenetic component 214 aligns the sequence read 304 such that the three CpG sites correlate to CpG sites 1, 2, and 3. The epigenetic component 214 thus generates information both on methylation status of all CpG sites on the cfDNA molecule 301 and the position in the human genome to which the CpG sites map. As shown, the CpG sites on sequence read 304 which were methylated are read as cytosines. In this example, the cytosines appear in the sequence read 304 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the epigenetic component 214 generates a methylation state vector 306 for the fragment cfDNA 301. In this example, the resulting methylation state vector 306 is <M1, U2, M3>, wherein M corresponds to a methylated CpG site, U corresponds to an unmethylated CpG site, and the subscript number corresponds to a position of each CpG site in the reference genome.

In another embodiment, after sequencing and alignment, the methylation status of an individual CpG site may be inferred from the count of methylated sequence reads “M” (methylated) and the count of unmethylated sequence reads “U” (unmethylated) at the cytosine residue in CpG context. A mean methylated CpG density (also called methylation density m) of specific loci in the plasma can be calculated using the equation: m=M/(M+U) where M is the count of methylated reads and U is the count of unmethylated reads at the CpG sites within the genetic locus. If there is more than one CpG site within a locus, then M and U correspond to the counts across the sites.

Besides sequencing, other techniques can be used to determine information regarding DNA methylation. In one embodiment, methylation profiling can be performed by methylation-specific PCR or methylation-sensitive restriction enzyme digestion followed by PCR or ligase chain reaction followed by PCR. In yet other embodiments, the PCR is a form of single molecule or digital PCR (B. Vogelstein et al. 1999 Proc Natl Acad Sci USA; 96: 9236-9241). In yet further embodiments, the PCR can be a real-time PCR. In other embodiments, the PCR can be multiplex PCR.

iii. Fragmentomic Component

Returning to FIG. 2, in an embodiment, a fragmentomic component 215 may analyze sequence fragments/reads to determine fragmentomic data. Fragmentomic data may include, for example, information regarding fragment size, nucleotide motifs at fragment ends, single-stranded jagged ends, genomic location of center point of the fragment length, genomic locations of fragment endpoints and/or any value indicating the endpoints of the fragment. The fragmentomic component 215 may be configured to analyze the sequence fragments/reads to determine one or more of: fragment size, end motif frequency, jagged end length, preferred end coordinates, center point coordinates, oriented end density, motif diversity score, a window protection score, cfDNA integrity, nucleosomal footprinting, combinations thereof, and the like. The fragmentomic data may be used as a fragmentomic signature. Fragmentomic data may be determined by any means known in the art. The fragmentomic data may be stored in the analysis datastore 218.

In an embodiment, the fragmentomic component 215 may be configured to determine an amount of the cell-free DNA fragments that have a particular size. The particular size can be a range. For example, a size range can be greater than or less than a size cutoff, e.g., 100 bp, 150 bp, or 200 bp. As other examples, the size range can be specified by a minimum and a maximum size, e.g., 50-80, 50-100, 50-150, 100-150, 100-200, 150-200, 150-230, 200-300, or 300-400 bases, as well as other ranges. The width of the size range can vary, e.g., to be 50, 100, 150, or 200 bases. As examples, the amount can be a raw count or be normalized, e.g., as a frequency using a total number of sequence reads or DNA fragments analyzed.

In an embodiment, the fragmentomic component 215 may be configured to determine an end motif for a sequence fragment/read and to determine an end motif frequency. An end motif relates to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

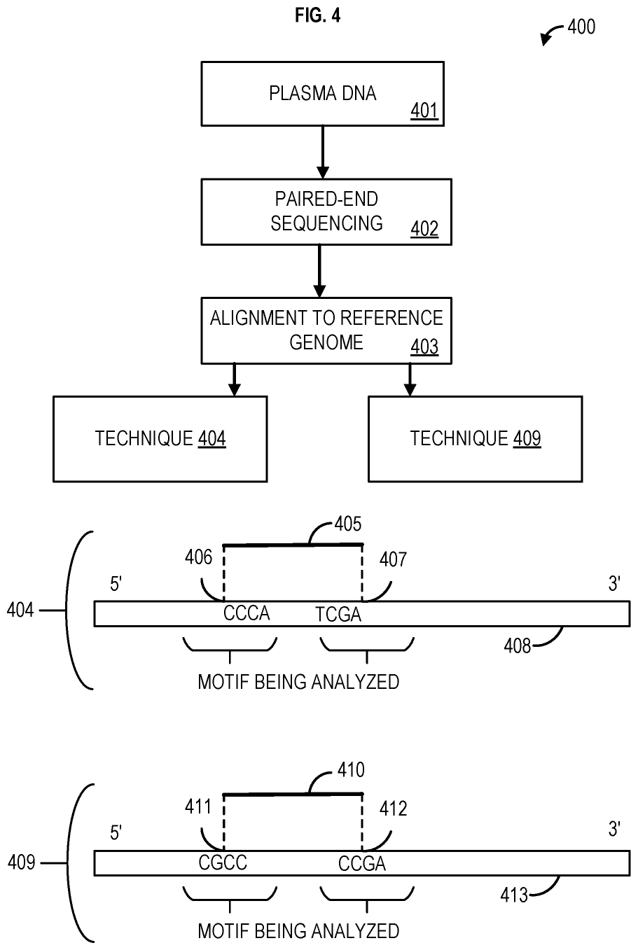

FIG. 4 shows examples for end motifs according to embodiments of the present disclosure. FIG. 4 depicts techniques to define 4-mer end motifs to be analyzed. In technique 404, the 4-mer end motifs are directly constructed from the first 4-bp sequence on each end of a plasma DNA molecule. For example, the first 4 nucleotides or the last 4 nucleotides of a sequenced fragment could be used. In technique 409, the 4-mer end motifs are jointly constructed by making use of the 2-mer sequence from the sequenced ends of fragments and the other 2-mer sequence from the genomic regions adjacent to the ends of that fragment. In other embodiments, other types of motifs can be used, e.g., 1-mer, 2-mer, 3-mer, 5-mer, 6-mer, 7-mer end motifs.

As shown in FIG. 4, a method 400 may begin with obtaining cell-free DNA fragments at step 401 via the laboratory system 202 and the sample collection and preparation pipeline 203 (e.g., using a purification process on a blood sample, such as by centrifuging). Besides plasma DNA fragments, other types of cell-free DNA molecules can be used, e.g., from serum, urine, saliva, and other samples mentioned herein. In one embodiment, the DNA fragments may be blunt-ended.

At step 402, the DNA fragments are subjected to paired-end sequencing via the sequencing pipeline 205. In some embodiments, the paired-end sequencing can produce two sequence reads from the two ends of a DNA fragment, e.g., 30-120 bases per sequence read. These two sequence reads can form a pair of reads for the DNA fragment (molecule), where each sequence read includes an ending sequence of a respective end of the DNA fragment. In other embodiments, the entire DNA fragment can be sequenced, thereby providing a single sequence read, which includes the ending sequences of both ends of the DNA fragment. The two ending sequences at both ends can still be considered paired sequence reads, even if generated together from a single sequencing operation.

At step 403, the fragmentomic component 215 may align the sequence reads to a reference genome. Such alignment is to illustrate different ways to define a sequence motif, and may not be used in some embodiments. For example, the sequences at the end of a fragment can be used directly without needing to align to a reference genome. However, alignment can be desired to have uniformity of an ending sequence, which does not depend on variations (e.g., SNPs) in the subject. For instance, the ending base could be different from the reference genome due to a variation or a sequencing error, but the base of in the reference may be the one counted. Alternatively, the base on the end of the sequence read can be used, so as to be tailored to the individual. The alignment procedure can be performed using various software packages, such as (but not limited to) BLAST, FASTA, Bowtie, BWA, BFAST, SHRiMP, SSAHA2, NovoAlign, and SOAP.

The method 400 may proceed to utilize technique 404 and/or technique 409 to further assess an end motif Technique 404 shows a sequence read of a sequence fragment 405, with an alignment to a genome 408. With the 5′ end viewed as the start, a first end motif 406 (CCCA) is at the start of sequence fragment 405. A second end motif 407 (TCGA) is at the tail of the sequence fragment 405. When analyzing the end predominance of cfDNA fragments, this sequence read would contribute to a C-end count for the 5′ end. Such end motifs might, in one embodiment, occur when an enzyme recognizes CCCA and then makes a cut just before the first C. If that is the case, CCCA will preferentially be at the end of the plasma DNA fragment. For TCGA, an enzyme might recognize it, and then make a cut after the A. When a count is determined for the A, this sequence read would contribute to an A-end count.

Technique 409 shows a sequence read of a sequenced fragment 410, with an alignment to a genome 413. With the 5′ end viewed as the start, a first end motif 411 (CGCC) has a first portion (CG) that occurs just before the start of sequence fragment 410 and a second portion (CC) that is part of the ending sequence for the start of sequenced fragment 410. A second end motif 412 (CCGA) has a first portion (GA) that occurs just after the tail of sequenced fragment 410 and a second portion (CC) that is part of the ending sequence for the tail of sequenced fragment 410. Such end motifs might, in one embodiment, occur when an enzyme recognizes CGCC and then makes a cut just before the G and the C. If that is the case, CC will preferentially be at the end of the plasma DNA fragment with CG occurring just before it, thereby providing an end motif of CGCC. As for the second end motif 164 (CCGA), an enzyme can cut between C and G. If that is the case, CC will preferentially be at the end of the plasma DNA fragment. For technique 409, the number of bases from the adjacent genome regions and sequenced plasma DNA fragments can be varied and are not necessarily restricted to a fixed ratio, e.g., instead of 2:2, the ratio can be 2:3, 3:2, 4:4, 2:4, etc.

The higher the number of nucleotides included in the cell-free DNA end signature, the higher the specificity of the motif because the probability of having 6 bases ordered in an exact configuration in the genome is lower than the probability of having 2 bases ordered in an exact configuration in the genome. Thus, the choice of the length of the end motif can be governed by the needed sensitivity and/or specificity of the intended use application.

As the ending sequence is used to align the sequence read to the reference genome, any sequence motif determined from the ending sequence or just before/after is still determined from the ending sequence. Thus, technique 409 makes an association of an ending sequence to other bases, where the reference is used as a mechanism to make that association. A difference between techniques 404 and 409 would be to which two end motifs a particular DNA fragment is assigned, which affects the particular values for the relative frequencies. But, the overall result (e.g., detecting a genetic disorder, determining efficacy of a dosage, monitoring activity of a nuclease, etc.) would not be affected by how the a DNA fragment is assigned to an end motif, as long as a consistent technique is used, e.g., for any training data to determine a reference value, as may occur using a machine learning model.

The counted numbers of DNA fragments having an ending sequence corresponding to a particular end motif (e.g., a particular base) may be counted (e.g., stored in an array in memory) to determine an amount of the particular end motif. The amount can be measured in various ways, such as a raw count or a frequency, where the amount is normalized. The normalization may be done using (e.g., dividing by) a total number of DNA fragments or a number in a specified group of DNA fragments (e.g., from a specified region, having a specified size, or having one or more specified end motifs). Differences in amounts of end motifs have been detected when a genetic disorder exists, as well as when an effective dose of an anticoagulant has been administered, as well as when the activity of a nuclease changes (e.g., increases or decreased).

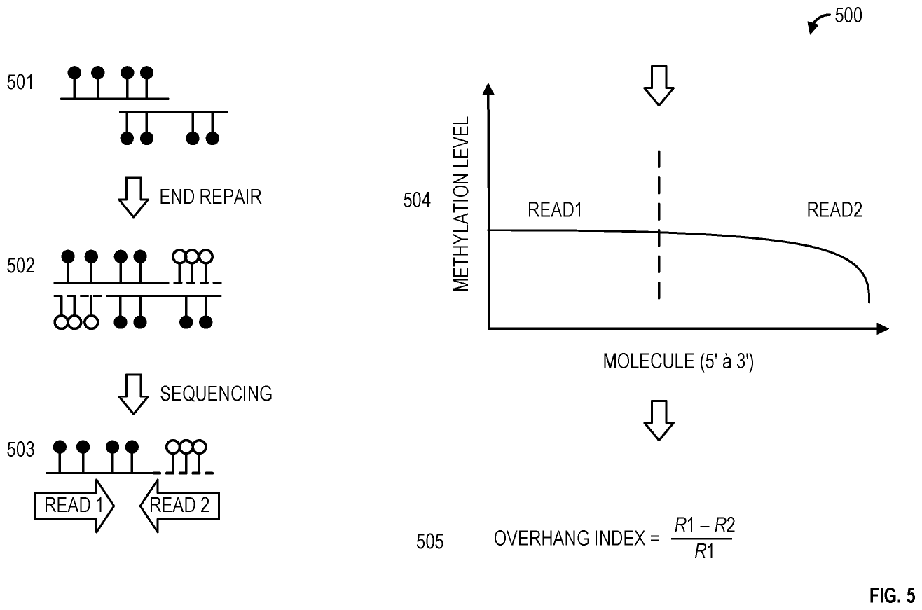

In an embodiment, the fragmentomic component 215 may be configured to determine a presence of a jagged end (e.g., an overhang) and an associated quantitative value. FIG. 5 illustrates one example showing how the degree of overhangs of cell-free DNA molecules (i.e., overhang index) can be determined. Diagrams 501, 502, and 503 include filled circles that represent methylated CpG sites, and unfilled circles that represent unmethylated CpG sites. Diagrams 502 and 503 include a dashed line that represents newly filled-up nucleotides. Diagram 503 includes an arrow indicative of the first read (read 1) in sequencing results and an arrow indicative of the secondary read (read 2). Graph 504 shows a graph of methylation level in read 1 and read 2 from 5′ to 3′ and an overhang index 250

$$( \frac{R 1 - R 2}{R 2} )$$

that comprises the following variables: R1 as the methylation level of read 1 and R2 as the methylation level of read 2.

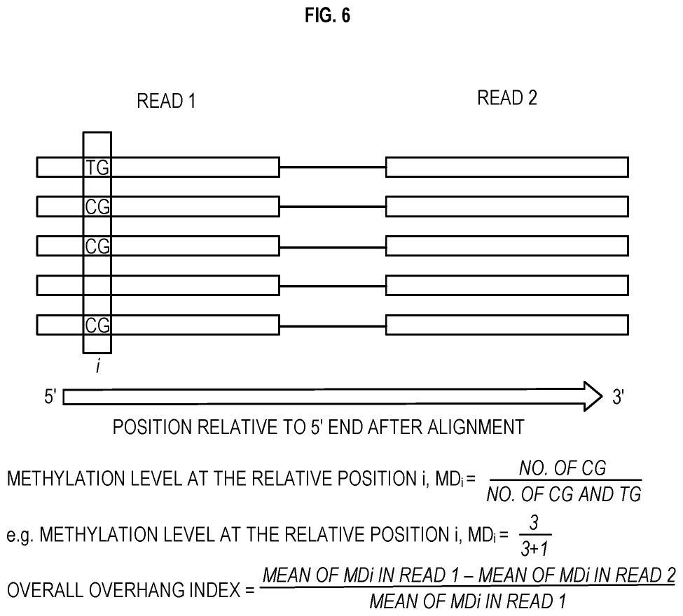

FIG. 6 is an illustration of the calculation of methylation levels along a DNA molecule after mapping to the human reference genome. All DNA molecules from the Watson and Crick strand may be stacked, respectively, according to relative positions and orientations after mapping to the human reference genome. The stacked molecules may be used for calculating an overall overhang index according to the positions relative to 5′ end in the alignment results as shown in FIG. 6.

The methylation level (VID) at a particular position i relative to the closest end (i.e., 5′ end for read 1) may be quantified by the ratio of the number of C's to the total number of C's and T's:

$${MD}_{i} = \frac{\# of CG}{\# of CG and TG} .$$

The first read (having 5′ end, i.e. read 1) may have a higher averaged methylation level than the second read (having 3′ end, i.e. read 2) because the 3′ gaps in the second read would be filled in by unmethylated C's which would be converted to T's in bisulfite sequencing results. An overall overhang index may be determined according to the following:

$$\frac{MEAN OF {MD}_{i} IN READ 1 - MEAN OF {MD}_{i} IN READ 2}{MEAN OF {MD}_{i} IN READ 1} .$$

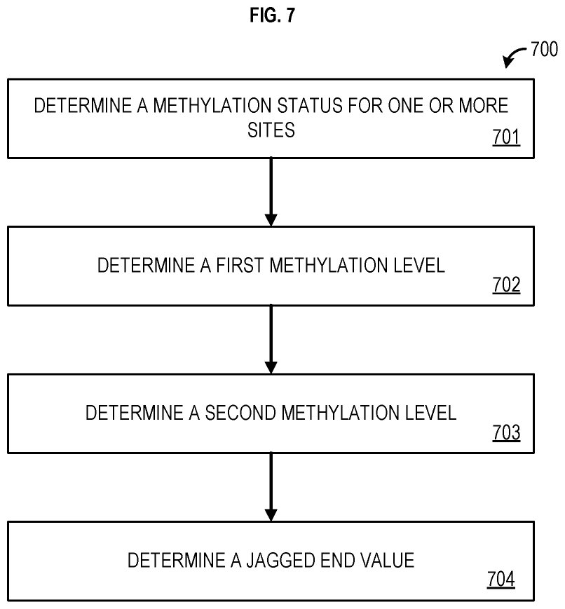

FIG. 7 shows a method 700 of determining an overhang index. A biological sample may include a plurality of nucleic acid molecules. The plurality of nucleic acid molecules may be cell-free. Each nucleic acid molecule of the plurality of nucleic acid molecules may be double-stranded with a first strand having a first portion and a second strand. The first portion of the first strand of at least some of the plurality of nucleic acid molecules may overhang the second strand, may not be hybridized to the second strand, and may be at a first end of the first strand.

At step 701, a methylation status of one or more sites of one or more strands may be determined. A first compound including one or more nucleotides may be hybridized to the first portion of the first strand for each nucleic acid molecule of the plurality of nucleic acid molecules. The first compound may be attached to a first end of the second strand to form an elongated second strand with a first end including the first compound. The first compound may include a first end not contacting the second strand. The one or more nucleotides may be unmethylated. In other implementations, certain nucleotides (e.g., cytosine) are all methylated, with the other nucleotides not being methylated. The first compound may be hybridized to the first portion one nucleotide at a time.

The first strand may be separated from the elongated second strand for each nucleic acid molecule of the plurality of nucleic acid molecules. A first methylation status for each of one or more first sites of the elongated second strand may be determined for each nucleic acid molecule of the plurality of nucleic acid molecules. The one or more first sites may be at the first end of the elongated second strand. A second methylation status for each of one or more second sites of the elongated second strand may optionally be determined for each nucleic acid molecule of the plurality of nucleic acid molecules. The one or more second sites may be at the second end of the elongated second strand. The one or more second sites may include the outermost sites at the second end of the elongated second strand. In some examples, the methylation status for the second sites may not need to be determined and may instead be assumed to be an average methylation status. The average methylation status may be known from a known frequency of methylated CpG sites in a particular region of the genome. In some instances, the average methylation status may be determined from reference samples taken from the same individual from which the biological sample is obtained and/or from other individuals.

At step 702, a first methylation level may be determined using the first methylation statuses for the plurality of elongated second strands at the one or more first sites. The first methylation level may be a mean or median of the first methylation statuses.

At step 703, a second methylation level may optionally be calculated using the second methylation statuses for the plurality of elongated second strands at the one or more second sites. The second methylation level may be a mean or median of the second methylation statuses. In some embodiments, the second methylation level may be assumed to be an average methylation level. The average methylation level may be based on a known frequency of methylated CpG sites in a particular region of the genome. In some instances, the average methylation level may be determined from reference samples taken from the same individual from which the biological sample is obtained and/or from other individuals. For example, the second methylation level may be assumed to be a value from 70% to 80%.

At step 704, an overhang index using the first methylation level and the second methylation level may be determined. A difference between the first methylation level and the second methylation level may be proportional to an average length of the first strands that overhang the second strands. Calculating the overhang index may be by calculating a difference between the first methylation level and the second methylation level and dividing the difference by the first methylation level (e.g., overall overhang index of FIG. 6).

In an embodiment, the fragmentomic component 215 may be configured to determine genomic locations of fragment endpoints. The fragmentomic component 215 may determine information about the two physical ends of DNA molecules. Both outer alignment coordinates of paired end data for which both reads aligned to the same chromosome and where reads have opposite orientations may be used as read starts. In cases where paired end data was converted to single read data by adapter trimming, both end coordinates of the single read alignment may be used as read starts. For coverage, all positions between the two (inferred) molecule ends, including these end positions, may be considered. It is expected that cfDNA fragment endpoints should cluster adjacent to nucleosome boundaries, while also being depleted on the nucleosome itself. To quantify this, a windowed protection scores (WPS) of a window size k may be defined as the number of molecules spanning a window minus those starting at any bases encompassed by the window. The determined WPS may be assigned to the center of the window. For molecules in the 35-80 bp range (short fraction), a window size of 16 may be used, for example, and, for molecules in the 120-180 bp (long fraction), a window size of 120 may be used, for example. High WPS values indicate increased protection of DNA from digestion; low values indicate that DNA is unprotected. Peak calls identify contiguous regions of elevated WPS.

Returning to FIG. 2, the results determined by the epigenetic component 214 and the fragmentomic component 215 may be associated with the sequence fragments and/or variants in the sequence data that were used to generate such results. And, in the instance of the sequence data being derived from known samples 201, the origin of the sequence fragments and/or variants may also be associated with the sequence data, the epigenetic data, and/or the fragmentomic data. For example, sequence data, epigenetic data, and fragmentomic data of sequence fragments and/or variants known to be tumor derived may be labeled as tumor derived and sequence data, epigenetic data, and fragmentomic data of sequence fragments and/or variants known to be non-tumor derived may be labeled as non-tumor derived. Moreover, further labels may be assigned, for example, cancer type, tissue type, and the like.

iv. Copy Number Component

The copy number component 216 may use the sequence fragments/reads to generate a chromosomal region of coverage. The copy number component 216 may divide the chromosomal regions into variable length windows or bins. A window or bin may be at least 5 kb, 10, kb, 25 kb, 30 kb, 35, kb, 40 kb, 50 kb, 60 kb, 75 kb, 100 kb, 150 kb, 200 kb, 500 kb, or 1000 kb. A window or bin may also have bases up to 5 kb, 10, kb, 25 kb, 30 kb, 35, kb, 40 kb, 50 kb, 60 kb, 75 kb, 100 kb, 150 kb, 200 kb, 500 kb, or 1000 kb. A window or bin may also be about 5 kb, 10, kb, 25 kb, 30 kb, 35, kb, 40 kb, 50 kb, 60 kb, 75 kb, 100 kb, 150 kb, 200 kb, 500 kb, or 1000 kb.

The copy number component 216 may normalize coverage by causing the window or bin to contain about the same number of mappable bases. In some cases, each window or bin in a chromosomal region may contain the exact number of mappable bases. In other cases, each window or bin may contain a different number of mappable bases. Additionally, each window or bin may be non-overlapping with an adjacent window or bin. In other cases, a window or bin may overlap with another adjacent window or bin. In some cases a window or bin may overlap by at least 1 bp, 2, bp, 3 bp, 4 bp, 5, bp, 10 bp, 20 bp, 25 bp, 50 bp, 100 bp, 200 bp, 250 bp, 500 bp, or 1000 bp. In other cases, a window or bin may overlap by up to 1 bp, 2, bp, 3 bp, 4 bp, 5, bp, 10 bp, 20 bp, 25 bp, 50 bp, 100 bp, 200 bp, 250 bp, 500. bp, or 1000 bp. In some cases a window or bin may overlap by about 1 bp, 2, bp, 3 bp, 4 bp, 5, bp, 10 bp, 20 bp, 25 bp, 50 bp, 100 bp, 200 bp, 250 bp, 500 bp, or 1000 bp.

In some cases, each of the window regions may be sized so they contain about the same number of uniquely mappable bases. The mappability of each base that comprise a window region is determined and used to generate a mappability file which contains a representation of fragments/reads from the references that are mapped back to the reference for each file. The mappability file contains one row per every position, indicating whether each position is or is not uniquely mappable.

Additionally, predefined windows, known throughout the genome to be hard to sequence, or contain a substantially high GC bias, may be filtered from the data set. For example, regions known to fall near the centromere of chromosomes (i.e., centromeric DNA) are known to contain highly repetitive sequences that may produce false positive results. These regions may be filtered out. Other regions of the genome, such as regions that contain an unusually high concentration of other highly repetitive sequences such as microsatellite DNA, may be filtered from the data set.

The number of windows analyzed may also vary. In some cases, at least 10, 20, 30, 40, 50, 100, 200, 500, 1000, 2000, 5,000, 10,000, 20,000, 50,000 or 100,000 windows are analyzed. In other cases, the number of widows analyzed is up to 10, 20, 30, 40, 50, 100, 200, 500, 1000, 2000, 5,000, 10,000, 20,000, 50,000 or 100,000 windows are analyzed.

The copy number component 216 may determine the read coverage for each window/bin region. This may be performed using either fragments/reads with barcodes, or without barcodes. In cases without barcodes, the previous mapping steps will provide coverage of different base positions. Sequence fragments/reads that have sufficient mapping and quality scores and fall within chromosome windows that are not filtered, may be counted. The number of coverage fragments/reads may be assigned a score per each mappable position.

In an embodiment, a quantitative measure related to sequencing read coverage is a measure indicative of the number of fragments/reads derived from a DNA molecule corresponding to a genetic locus (e.g., a particular position, base, region, gene or chromosome from a reference genome). In order to associate fragments/reads to a genetic locus, the fragments/reads can be mapped or aligned to the reference. Software to perform mapping or aligning (e.g., Bowtie, BWA, mrsFAST, BLAST, BLAT) can associate a sequencing read with a genetic locus. During the mapping process, particular parameters can be optimized. Non-limiting examples of optimization of the mapping processing can include masking repetitive regions; employing mapping quality (e.g., MAPQ) score cut-offs; using different seed lengths to generate alignments; and limiting the edit distance between positions of the genome.

Quantitative measures associated with sequencing read coverage can include counts of fragments/reads associated with a genetic locus. In some cases, the counts are transformed into new metrics to mitigate the effects of differing sequencing depth, library complexity, or size of the genetic locus. Exemplary metrics are Read Per Kilobase per Million (RPKM), Fragments Per Kilobase per Million (FPKM), Trimmed Mean of M values (TMM), variance stabilized raw counts, and log transformed raw counts. Other transformations are also known to those of skill in the art that may be used for particular applications.

Quantitative measures can be determined using numbers of fragment/read families or collapsed fragments/reads, wherein each read family or collapsed read corresponds to an initial template DNA molecule. Methods to collapse and quantify read families are found in PCT/US2013/058061 and PCT/US2014/000048, each of which is herein incorporated by reference in its entirety. In particular, quantifying read families and/or collapsing methods can be employed that use barcodes and sequence information from the sequencing read to sort fragments/reads into families, such that each family shares barcode sequences and at least a portion of the sequencing read sequence and/or the same genomic coordinates when mapped to a reference sequence. Each family is then, for the majority of the families, derived from a single initial template DNA molecule. Counts derived from mapping sequences from families can be referred to as “unique molecular counts” (UMCs). In some cases, determining a quantitative measure related to sequencing read coverage comprises normalizing UMCs by a metric related to library size to provide normalized UMCs (“normalized UMCs”). Exemplary methods are dividing the UMC of a genetic locus by the sum of all UMCs; dividing the UMC of a genetic locus by the sum of all autosomal UMCs. When comparing multiple sequencing read data sets, UMCs can, for example, be normalized by the median UMCs of the genetic loci of the two sequencing read data sets. In some cases, the quantitative measure related to sequencing read coverage can be normalized UMCs that are further normalized as follows: (i) normalized UMCs are determined for corresponding genetic loci from sequencing fragments/reads derived from training samples; (ii) for each genetic locus, normalized UMCs of the sample are normalized by the median of the normalized UMCs of the training samples at the corresponding loci, thereby providing Relative Abundances (RAs) of genetic loci.

Consensus sequences can identified based on their sequences, for example by collapsing sequencing fragments/reads based on identical sequences within the first 5, 10, 15, 20, or 25 bases. In some cases, collapsing allows for 1 difference, 2 differences, 3 differences, 4 differences, or 5 differences in the fragments/reads that are otherwise identical. In some cases, collapsing uses the mapping position of the read, for example the mapping position of the initial base of the sequencing read. In some cases, collapsing uses barcodes, and sequencing fragments/reads that share barcode sequences are collapsed into a consensus sequence. In some cases, collapsing uses both barcodes and the sequence of the initial template molecules. For example, all fragments/reads that share a barcode and map to the same position in the reference genome can be collapsed. In another example, all fragments/reads that share a barcode and a sequence of the initial template molecule (or a percentage identity to a sequence of the initial template molecule) can be collapsed.

In some cases, quantitative measures of sequencing read coverage are determined for specific sub-regions of a genome. Regions can be bins, genes of interest, exons, regions corresponding to sequence probes, regions corresponding to primer amplification products, or regions corresponding to primer binding sites. In some cases, sub-regions of the genome are regions corresponding to sequence capture probes. A read can map to a region corresponding to the sequence capture probe if at least a portion of the read maps at least a portion of the region corresponding to the sequence capture probe. A read can map to a region corresponding to the sequence capture probe if at least a portion of the read maps to the majority of the region corresponding to the sequence capture probe. A read can map to a region corresponding to the sequence capture probe if at least a portion of the read maps across the center point of the region corresponding to the sequence capture probe.

In another embodiment involving barcodes, all sequences with the same barcode, physical properties or combination of the two may be collapsed into one read, as they are all derived from the sample parent molecule to reduce biases which may have been introduced during amplification. For example, if one molecule is amplified 10 times but another is amplified 1000 times, each molecule is only represented once after collapse thereby negating the effect of uneven amplification. Only fragments/reads with unique barcodes may be counted for each mappable position and influence the assigned score.

Consensus sequences can be generated from families of sequence fragments/reads by any method known in the art. Such methods include, for example, linear or non-linear methods of building consensus sequences (such as voting, averaging, statistical, maximum a posteriori or maximum likelihood detection, dynamic programming, Bayesian, hidden Markov or support vector machine methods, etc.) derived from digital communication theory, information theory, or bioinformatics.

After the sequence read coverage has been determined, a stochastic modeling algorithm may be applied to convert the normalized nucleic acid sequence read coverage for each window/bin region to the discrete copy number states. In some cases, this algorithm may comprise one or more of the following: Hidden Markov Model, dynamic programming, support vector machine, Bayesian network, trellis decoding, Viterbi decoding, expectation maximization, Kalman filtering methodologies and neural networks. The discrete copy number states of each window region can be utilized to identify copy number variation in the chromosomal regions. In some cases, all adjacent window/bin regions with the same copy number can be merged into a segment to report the presence or absence of copy number variation state. In some cases, various windows/bins can be filtered before they are merged with other segments. The copy number variation may be stored in the analysis datastore 218 and/or reported as graph, indicating various positions in the genome and a corresponding increase or decrease or maintenance of copy number variation at each respective position. Additionally, copy number variation may be used to report a percentage score indicating how much disease material (or nucleic acids having a copy number variation) exists in the cell free polynucleotide sample.

v. Variant Caller Component

A variant caller 217 may retrieve/receive data from the analysis datastore 218. For example, the variant caller 217 may retrieve/receive data representing a plurality of sequence fragments/reads. The plurality of sequence fragments/reads may be analyzed to determine one or more variants. Variants may include, for example, single nucleotide variants (SNVs), indels, fusions, and copy number variation. Any known technique for variant calling may be used. In an embodiment, nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Any data analyzed, determined, and/or output by the sequence analysis pipeline 212 may be stored in the analysis datastore 218. Generally speaking, the processor 220 may implement (be programmed by) various components of the sequence analysis pipeline 212, such as the sequence quality control component 213, the epigenetic component 214, the fragmentomic component 215, the copy number component 216, the variant caller 217, and/or other components. Alternatively, it should be noted that these components of the sequence analysis pipeline 212 may include a hardware module. Although illustrated separately for convenience, one or more of the various components or instructions, such as the sequence quality control component 213, the epigenetic component 2114, the fragmentomic component 215, the copy number component 216, and/or the variant caller 217 may be integrated with one another.

The computer system 210 may exchange data with a computer system 224 using a network 223. For example, the computer system 224 may retrieve data from the analytics datastore 218. The computer system 224 may be configured for generating a predictive model (e.g., a classifier) and/or for utilizing a predictive model to determine an origin of a sequence fragment and/or variant.

h. Predictive Models

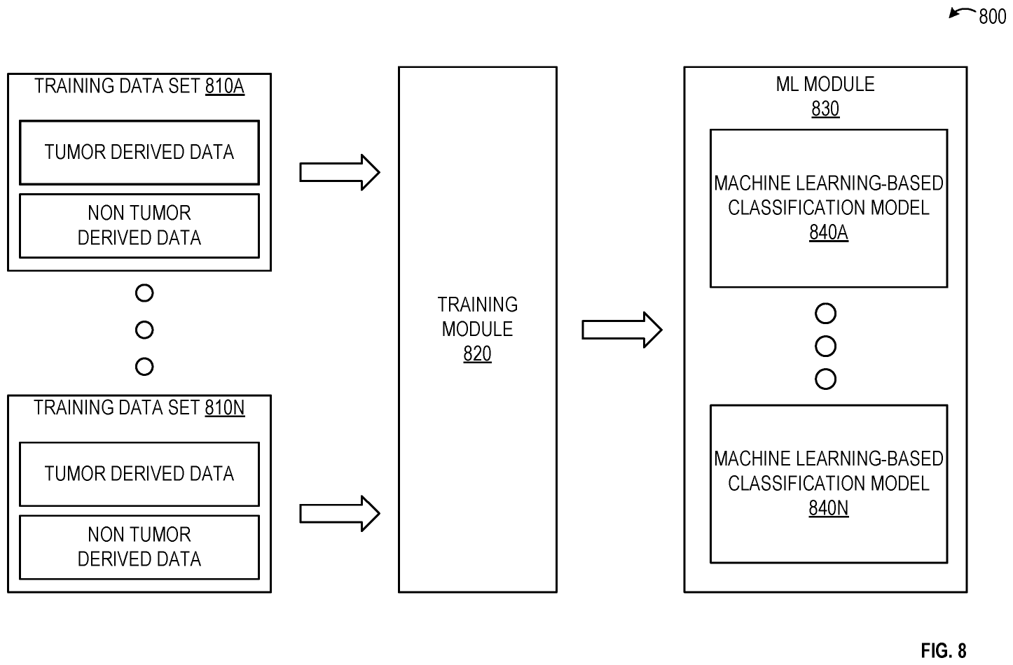

Turning now to FIG. 8, additional methods are described for generating a predictive model (e.g., a classifier). The methods described may use machine learning (“ML”) techniques to train, based on an analysis of one or more training data sets 810 by a training module 820, at least one ML module 830 that is configured to classify sequence fragments and/or variants in plasma as tumor origin or non-tumor origin, which can be from clonal hematopoiesis or biological noise.

The training data set 810 may comprise tumor derived and non-tumor derived (e.g., cancer/non-cancer) bodily fluid (e.g., blood, plasma, serum, cerebrospinal fluid, urine) sample data. The sample data may comprise sequence data which may comprise sequence information for one or more sequence fragments/reads and/or variants. The sample data may comprise epigenetic data. The epigenetic data may include, for example, information regarding DNA methylation, histone states or modifications, inflammation-mediated cytosine damage products, protein binding, or other molecular states reflected in the nucleic acid fragment analyzed that are not ascertained solely from the nucleotide base sequence, e.g., the methylation status of give base or set bases. The sample data may comprise fragmentomic data. The fragmentomic data may include, for example, information regarding fragment mapped starts and stops positions (correlated with nucleosome positions), fragment length and associated nucleosome occupancy. In an embodiment, the origin (tumor derived and non-tumor derived) of the sequence fragments/reads and/or variants in the sequence data may also be associated with the sequence data, the epigenetic data, and/or the fragmentomic data. For example, sequence data, epigenetic data, and fragmentomic data of sequence fragments/reads and/or variants known to be tumor derived may be labeled as tumor derived and sequence data, epigenetic data, and fragmentomic data of sequence fragments and/or variants known to be non-tumor derived may be labeled as non-tumor derived. Moreover, further labels may be assigned, for example, cancer type, tissue type, and the like.

A subset of the tumor derived/non-tumor derived sample data may be randomly assigned to the training data set 810 or to a testing data set. In some implementations, the assignment of data to a training data set or a testing data set may not be completely random. In this case, one or more criteria may be used during the assignment. In general, any suitable method may be used to assign the data to the training or testing data sets, while ensuring that the data distributions are somewhat similar in the training data set and the testing data set.

The training module 820 may train the ML module 830 by extracting a feature set from the tumor derived/non-tumor derived sample data in the training data set 810 according to one or more feature selection techniques. The training module 820 may train the ML module 830 by extracting a feature set from the training data set 810 that includes statistically significant features.

The training module 820 may extract a feature set from the training data set 810 in a variety of ways. The training module 820 may perform feature extraction multiple times, each time using a different feature-extraction technique. In an example, the feature sets generated using the different techniques may each be used to generate different machine learning-based classification models 840. For example, the feature set with the highest quality metrics may be selected for use in training. The training module 820 may use the feature set(s) to build one or more machine learning-based classification models 840A-840N that are configured to classify an origin as tumor or non-tumor for a new variant (e.g., with an unknown origin).

The training data set 810 may be analyzed to determine any dependencies, associations, and/or correlations between features and the experimental parameters in the training data set 810. The identified correlations may have the form of a list of features. The term “feature,” as used herein, may refer to any characteristic of an item of data that may be used to determine whether the item of data falls within one or more specific categories. By way of example, the features described herein may comprise any data and/or calculated values described herein, including: frequency of observance of a genetic variant among samples of particular cancer type, including hematological malignancies; prevalence of variants in plasma, tumor tissue, or white blood cells; methylation state vectors; methylation densities; fragment sizes; fragment size distributions; end motifs; end motif frequencies; jagged end presence; overhang indexes; genomic location of center point of the fragment length, genomic locations of fragment endpoints, any value indicating the endpoints of the fragment; windowed protection scores; combinations thereof and the like.

A feature selection technique may comprise one or more feature selection rules. The one or more feature selection rules may comprise a feature occurrence rule. The feature occurrence rule may comprise determining which features in the training data set 810 occur over a threshold number of times and identifying those features that satisfy the threshold as features.

A single feature selection rule may be applied to select features or multiple feature selection rules may be applied to select features. The feature selection rules may be applied in a cascading fashion, with the feature selection rules being applied in a specific order and applied to the results of the previous rule. For example, the feature occurrence rule may be applied to the training data set 810 to generate a first list of features. A final list of features may be analyzed according to additional feature selection techniques to determine one or more feature groups (e.g., groups of features that may be used to classify a sequence fragment/read and/or variant as tumor derived or non-tumor derived). Any suitable computational technique may be used to identify the feature groups using any feature selection technique such as filter, wrapper, and/or embedded methods. One or more feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable.

As another example, one or more feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. As an example, forward feature selection may be used to identify one or more feature groups. Forward feature selection is an iterative method that begins with no feature in the machine learning model. In each iteration, the feature which best improves the model is added until an addition of a new variable does not improve the performance of the machine learning model. As an example, backward elimination may be used to identify one or more feature groups. Backward elimination is an iterative method that begins with all features in the machine learning model. In each iteration, the least significant feature is removed until no improvement is observed on removal of features. Recursive feature elimination may be used to identify one or more feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 820 has generated a feature set(s), the training module 820 may generate a machine learning-based classification model 840 based on the feature set(s). A machine learning-based classification model may refer to a complex mathematical model for data classification that is generated using machine-learning techniques. In one example, the machine learning-based classification model 840 may include a map of support vectors that represent boundary features. By way of example, boundary features may be selected from, and/or represent the highest-ranked features in, a feature set.

The training module 820 may use the feature sets determined or extracted from the training data set 810 to build a machine learning-based classification model 840A-840N. In some examples, the machine learning-based classification models 840A-840N may be combined into a single machine learning-based classification model 840. Similarly, the ML module 830 may represent a single classifier containing a single or a plurality of machine learning-based classification models 840 and/or multiple classifiers containing a single or a plurality of machine learning-based classification models 840.

The features may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting ML module 830 may comprise a decision rule or a mapping for each feature to determine tumor/non-tumor origin for a variant.

In an embodiment, the training module 820 may train the machine learning-based classification models 840 as a convolutional neural network (CNN). The CNN comprises at least one convolutional feature layer and three fully connected layers leading to a final classification layer (softmax). The final classification layer may finally be applied to combine the outputs of the fully connected layers using softmax functions as is known in the art.

The feature(s) and the ML module 830 may be used to predict the tumor derived or non-tumor derived origin of sequence fragments/reads and/or variants in the testing data set. In one example, the prediction result for each sequence fragment/read and/or variant may include a confidence level that corresponds to a likelihood or a probability that a sequence fragment/read and/or variant in the testing data set is associated with tumor origin or non-tumor origin. The confidence level may be a value between zero and one. In one example, when there are two statuses (e.g., tumor origin and non-tumor origin), the confidence level may correspond to a value p, which refers to a likelihood that a particular variant belongs to the first status (e.g., tumor origin). In this case, the value 1−p may refer to a likelihood that the particular variant belongs to the second status (e.g., non-tumor origin). In general, multiple confidence levels may be provided for each variant in the testing data set and for each feature when there are more than two statuses. A top performing feature may be determined by comparing the result obtained for each test variant with the known tumor/non-tumor origin for each test variant. In general, the top performing feature will have results that closely match the known tumor/non-tumor origin statuses. The top performing feature(s) may be used to predict/classify the tumor/non-tumor origin status of a given variant.

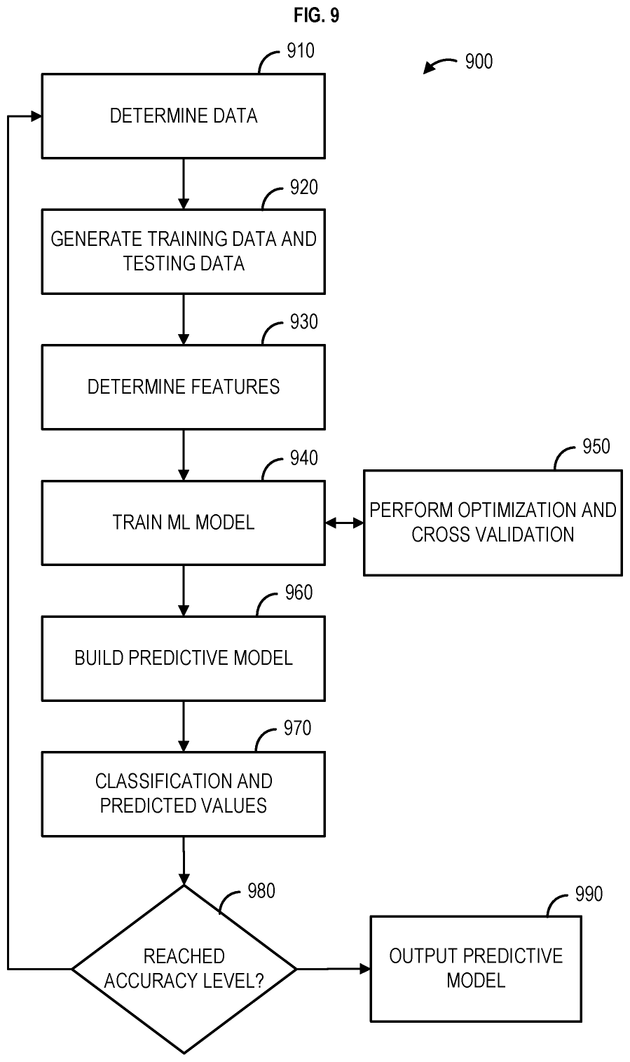

FIG. 9 is a flowchart illustrating an example training method 900 for generating the ML module 830 using the training module 820. The training module 820 can implement supervised, unsupervised, and/or semi-supervised (e.g., reinforcement based) machine learning-based classification models 840. The method 900 illustrated in FIG. 9 is an example of a supervised learning method; variations of this example of training method are discussed below, however, other training methods can be analogously implemented to train unsupervised and/or semi-supervised machine learning models.

The training method 900 may determine (e.g., access, receive, retrieve, etc.) data at step 910. The data may comprise tumor derived/non-tumor derived bodily fluid sample data. The data may comprise sequence data, epigenetic data, and/or fragmentomic data for one or more sequence fragments reads and/or variants, each sequence fragment/read and/or variant having an assigned tumor derived or non-tumor derived origin status.

The training method 900 may generate, at step 920, a training data set and a testing data set. The training data set and the testing data set may be generated by randomly assigning data to either the training data set or the testing data set. In some implementations, the assignment of computation parameters and associated experimental parameters as training or testing data may not be completely random. As an example, a majority of the computation parameters and associated experimental parameters may be used to generate the training data set. For example, 75% of the computation parameters and associated experimental parameters may be used to generate the training data set and 25% may be used to generate the testing data set. In another example, 80% of the computation parameters and associated experimental parameters may be used to generate the training data set and 20% may be used to generate the testing data set.

The training method 900 may determine (e.g., extract, select, etc.), at step 930, one or more features that can be used by, for example, a classifier to differentiate among different classification of tumor derived vs. non-tumor derived status. As an example, the training method 900 may determine a set of features from the tumor derived/non-tumor derived bodily fluid sample data. In a further example, a set of features may be determined from data that is different than the tumor derived/non-tumor derived bodily fluid sample data in either the training data set or the testing data set. Such other data may be used to determine an initial set of features, which may be further reduced using the training data set.

The training method 900 may train one or more machine learning models using the one or more features at step 940. In one example, the machine learning models may be trained using supervised learning. In another example, other machine learning techniques may be employed, including unsupervised learning and semi-supervised. The machine learning models trained at 940 may be selected based on different criteria depending on the problem to be solved and/or data available in the training data set. For example, machine learning classifiers can suffer from different degrees of bias. Accordingly, more than one machine learning model can be trained at 940, optimized, improved, and cross-validated at step 950.

The training method 900 may select one or more machine learning models to build a predictive model at 960. The predictive model may be evaluated using the testing data set. The predictive model may analyze the testing data set and generate predicted tumor/non-tumor origin statuses at step 970. Predicted tumor/non-tumor origin may be evaluated at step 980 to determine whether such values have achieved a desired accuracy level. Performance of the predictive model may be evaluated in a number of ways based on a number of true positives, false positives, true negatives, and/or false negatives classifications of the plurality of data points indicated by the predictive model.

For example, the false positives of the predictive model may refer to a number of times the predictive model incorrectly classified a sequence fragment/read and/or variant as tumor origin that was in reality non-tumor origin. Conversely, the false negatives of the predictive model may refer to a number of times the machine learning model classified a sequence fragment/read and/or variant as non-tumor origin when, in fact, the sequence fragment/read and/or variant was tumor origin. True negatives and true positives may refer to a number of times the predictive model correctly classified one or more sequence fragment/read and/or variant. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives. When such a desired accuracy level is reached, the training phase ends and the predictive model (e.g., the ML module 830) may be output at step 990; when the desired accuracy level is not reached, however, then a subsequent iteration of the training method 900 may be performed starting at step 910 with variations such as, for example, considering a larger collection of data.

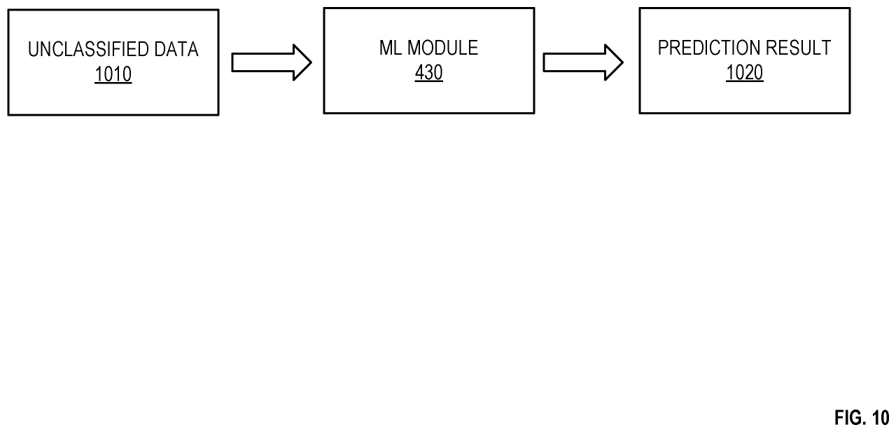

FIG. 10 is an illustration of an exemplary process flow for using a machine learning-based classifier to classify a sequence fragment/read and/or variant as tumor origin or non-tumor origin. As illustrated in FIG. 10, sequence data, epigenetic data, and/or fragmentomic data for an unclassified sequence fragment/read and/or variant 1010 may be provided as input to the ML module 830. The ML module 830 may process the sequence data, epigenetic data, and/or fragmentomic data for the unclassified sequence fragment/read and/or variant 1010 using a machine learning-based classifier(s) to arrive at a prediction result 1020. The prediction result 1020 may identify one or more characteristics of the sequence data, epigenetic data, and/or fragmentomic data for an unclassified sequence fragment/read and/or variant 1010. For example, the classification result 1020 may identify the origin status of the sequence fragment/read and/or variant 1010 (e.g., whether the sequence fragment/read and/or variant is tumor origin or non-tumor origin). Thus, in an embodiment, disclosed is a method implemented using a network-based computer system comprising one or more processors, a network interface, and one or more memories, the method comprising retrieving, by the computer system, sequence data, epigenetic data, and/or fragmentomic data having an indicated tumor derived origin or non-tumor derived origin status; and training, by the one or more processors, a machine-learning model by fitting one or more models to the sequence data, epigenetic data, and/or fragmentomic data, wherein each of the one or more models is configured to receive as input sequence data, epigenetic data, and/or fragmentomic data of an individual, and provide as output a prediction of the individual having or developing a tumor.

i. Example Methods

In some aspects, this disclosure provides methods of coupling somatic genomic information with epigenetic signatures (e.g. methylation profiles, fragmentomics, etc.) which provide additional genomic signal to aid in the bioinformatic exclusion of background clonal hematopoiesis of indeterminate potential (CHIP) variants to deterministically call tumor or in known CHIP genes. In some embodiments, the methylation and fragmentation profiles of normal white blood cells exhibiting CHIP are differentiated from their pathogenic tumor counterparts. In certain embodiments, incorporation of targeted hybridization panels investigating known methylation sites or other epigenetic sites in genes of likely CHIP interference (e.g., DNMT3A, TP53, LRP1B, KRAS, etc.) in the NGS workflow provides orthogonal information to adjudicate CHIP. Similarly, incorporation of bioinformatic modules analyzing the ctDNA fragment distribution of genes known to exhibit a high prevalence CHIP are used as orthogonal information to generate CHIP adjudication callers in some embodiments. The combination of known CHIP prevalence genes or other genomic regions and epigenetic profiles (e.g., methylation profiles, ctDNA fragment distributions (e.g., fragmentomics), bi-sulfide sequencing, and/or the like) provide technological solutions to improve the efficacy of diagnostics.

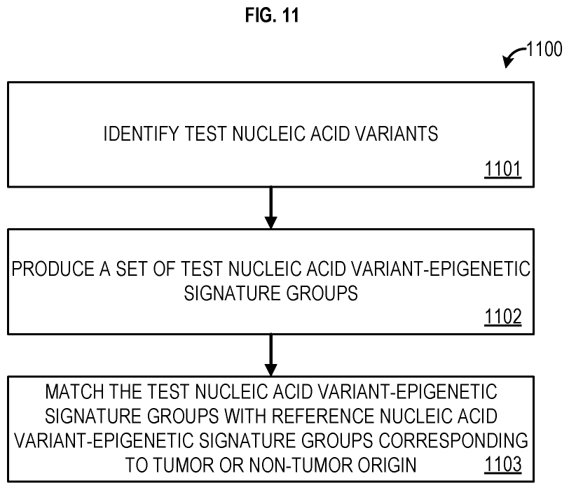

To illustrate, FIG. 11 is a flow chart that schematically depicts exemplary method steps of differentiating tumor and clonal hematopoiesis of indeterminate potential (CHIP) origin nucleic acid variants from one another in a test sample obtained from a test subject using a computer according to some embodiments of the invention. As shown, method 1100 includes identifying nucleic acid variants in a set of targeted genomic regions from sequence information obtained from nucleic acids in the test sample to produce a set of identified test nucleic acid variants (step 1101). The method also includes identifying at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups (step 1102). In some embodiments, the epigenetic signature, for example—methylation signature, may be determined based on the methods and systems disclosed in PCT Application No. PCT/US2021/025201. The method also includes matching given test nucleic acid variant-epigenetic signature groups in the set of test nucleic acid variant-epigenetic signature groups with reference nucleic acid variant-epigenetic signature groups corresponding to tumor origin nucleic acid variants or with reference nucleic acid variant-epigenetic signature groups corresponding to CHIP origin nucleic acid variants, thereby differentiating the tumor and the CHIP origin nucleic acid variants from one another in the test sample obtained from the test subject (step 1103). In some embodiments, method 1100 also includes using at least one trained classifier to differentiate tumor and CHIP origin nucleic acid variants from one another in the set of test nucleic acid variant-epigenetic signature groups to produce a set of differentiated tumor and CHIP origin nucleic acid variants present in the test sample. In some embodiments, the method also includes administering at least one therapy to the test subject based upon one or more of the differentiated tumor origin nucleic acid variants in the set of differentiated tumor and CHIP origin nucleic acid variants present in the test sample to thereby treat the cancer in the test subject.

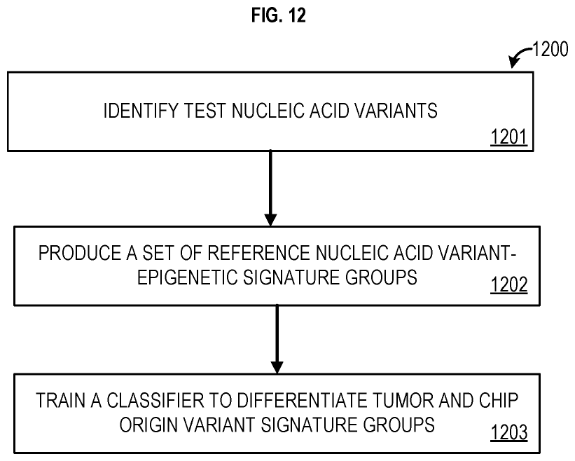

To illustrate, FIG. 12 is a flow chart that schematically depicts exemplary method steps of generating a trained classifier using a computer according to some embodiments of the invention. As shown, method 1200 includes identifying nucleic acid variants in at least one set of targeted genomic regions from sequence information obtained from nucleic acids in a plurality of reference samples to produce a set of identified reference nucleic acid variants (step 1201). Method 1200 also includes identifying at least one epigenetic signature corresponding to a given nucleic acid variant for a plurality of the identified reference nucleic acid variants in the set of identified reference nucleic acid variants from epigenetic information obtained from the nucleic acids in the reference samples to produce a set of reference nucleic acid variant-epigenetic signature groups (step 1202). Method 1200 also includes training a machine learning algorithm using at least a portion of the set of reference nucleic acid variant-epigenetic signature groups to create at least one trained classifier that is configured to classify one or more test nucleic acid variant-epigenetic signature groups as comprising tumor and/or clonal hematopoiesis of indeterminate potential (CHIP) origin nucleic acid variants (step 1203).

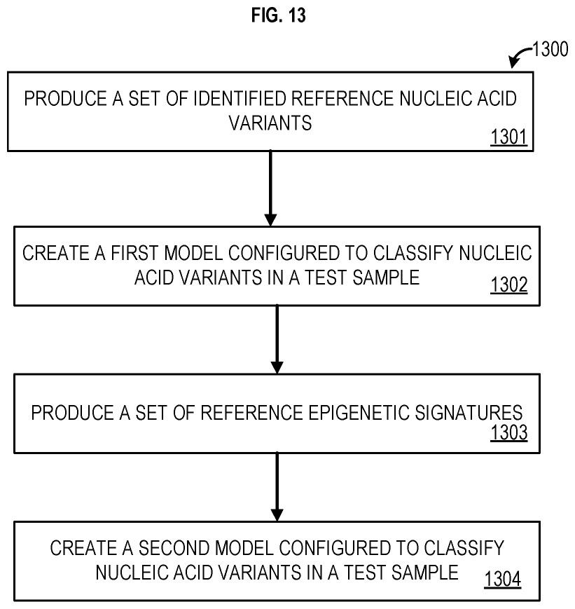

To further illustrate, FIG. 13 is a flow chart that schematically depicts exemplary method steps of generating a trained classifier using a computer according to some embodiments of the invention. As shown, method 1300 includes identifying nucleic acid variants in at least one set of targeted genomic regions from sequence information obtained from nucleic acids in a plurality of reference samples to produce a set of identified reference nucleic acid variants (step 1301). Method 1300 also includes training a machine learning algorithm using at least a portion of the set of identified reference nucleic acid variants to create at least a first model that is configured to classify nucleic acid variants in the set of targeted genomic regions from sequence information obtained from nucleic acids in a test sample to produce a set of identified test nucleic acid variants (step 1302). Method 1300 also includes identifying at least one epigenetic signature corresponding to a given nucleic acid variant for a plurality of the reference identified nucleic acid variants in the set of identified reference nucleic acid variants from epigenetic information obtained from the nucleic acids in the reference samples to produce a set of reference epigenetic signatures (step 1303). Method 1300 also includes training the machine learning algorithm using at least a portion of the set of reference epigenetic signatures to create at least a second model that is configured to differentiate tumor and clonal hematopoiesis of indeterminate potential (CHIP) origin nucleic acid variants from one another in the set of test nucleic acid variant-epigenetic signature groups to produce a set of identified test nucleic acid variants (step 1304).

In some embodiments, the set of test nucleic acid variant-epigenetic signature groups comprises at least first and second members that comprise identical nucleic acid variants and different corresponding epigenetic signatures. In some of these embodiments, the different corresponding epigenetic signatures comprise differing epigenetic states or statuses exhibited by one or more epigenetic loci in a given targeted genomic region. In some of these embodiments, the different corresponding epigenetic signatures comprise differing cell-free nucleic acid (cfNA) fragment length, position, and/or endpoint density distributions. In some embodiments, the set of test nucleic acid variant-epigenetic signature groups comprises at least first and second members that comprise different nucleic acid variants and identical corresponding epigenetic signatures.

In some embodiments, the matching step comprises using at least one trained classifier to differentiate the tumor and the CHIP origin nucleic acid variants from one another in the test sample obtained from the test subject. In some embodiments, the set of identified nucleic acid variants comprises somatic nucleic acid variants. In some embodiments, a given targeted genomic region comprises two or more nucleic acid variant loci. In some embodiments, the set of test nucleic acid variant-epigenetic signature groups comprises at least one member that comprises one or more nucleic acid variants and one or more corresponding epigenetic signatures that are from different genomic regions in the set of set of targeted genomic regions. In some embodiments, the set of test nucleic acid variant-epigenetic signature groups comprises at least one member that comprises one or more nucleic acid variants and one or more corresponding epigenetic signatures that are in an identical genomic region in the set of set of targeted genomic regions. In some embodiments, the plurality of targeted genomic regions comprise one or more genes selected from the group consisting of: DNAMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET. In some embodiments, the nucleic acids in the sample comprise cell-free nucleic acid (cfNA) fragments and/or nucleic acid molecules obtained from one or more tissues or cells in the sample. In some embodiments, the epigenetic signature comprises a cfNA fragment length, position, and/or endpoint density distribution.

In some embodiments, the epigenetic signature comprises an epigenetic state or status exhibited by one or more epigenetic loci in a given targeted genomic region. In some embodiments, the epigenetic state or status comprises a presence or absence of methylation, hydroxymethylation, acetylation, ubiquitylation, phosphorylation, sumoylation, ribosylation, citrullination, and/or a histone post-translational modification or other histone variation. In some embodiments, the method further includes disregarding differentiated CHIP origin nucleic acid variants from further analysis. In some embodiments, the method further includes generating at least one report that lists the tumor and CHIP origin nucleic acid variants differentiated from one another in the test sample.

In some embodiments, the method further includes identifying at least one cancer type associated with the differentiated tumor origin nucleic acid variants. In some embodiments, the method further includes administering at least one therapy to the test subject to treat the identified cancer type. In some embodiments, the method further includes administering at least one therapy to the test subject based upon one or more of the differentiated tumor origin nucleic acid variants. In some embodiments, one or more cells comprise the nucleic acids in the test sample.

In some embodiments, the method further includes identifying, by the computer, nucleic acid variants in the set of targeted genomic regions from sequence information obtained from nucleic acids in a test sample obtained from a test subject to produce a set of identified test nucleic acid variants, identifying, by the computer, at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups, and using the trained classifier to differentiate the tumor and the CHIP origin nucleic acid variants in the set of test nucleic acid variant-epigenetic signature groups from one another in the test sample obtained from the test subject. In some embodiments, the second model is a further trained version of the first model. In some embodiments, the set of reference nucleic acid variant-epigenetic signature groups comprises prevalence data for epigenetic signatures corresponding to given nucleic acid variants in the set of identified reference nucleic acid variants.

In some embodiments, identifying the at least one epigenetic signature corresponding to a given nucleic acid variant comprises: determining epigenetic rates corresponding to the given nucleic acid variant, wherein at least a first epigenetic rate is generated from a first sample obtained from a given subject at a first time point, and at least a second epigenetic rate is generated from a second sample obtained from the given subject at a second time point that differs from the first time point; adjusting at least one epigenetic rate threshold based on at least the first epigenetic rate to produce an adjusted epigenetic rate threshold; and using the adjusted epigenetic rate threshold to identify the epigenetic signature. In some embodiments, the first and second sample samples comprise test samples. In some embodiments, the first and second sample samples comprise reference samples. In some embodiments, the first sample comprises a tumor tissue sample. In some embodiments, the second sample comprises a bodily fluid sample. Some embodiments include using epigenetic rates to identify tumor fractions in samples. Certain embodiments optionally include determining a plurality of epigenetic rates for a plurality of genomic regions of a first sample; determining a likelihood of a tumor fraction for one or more of the plurality of genomic regions in a second sample based on a predetermined set of epigenetic rates of the plurality of genomic regions of the second sample, a set of epigenetic characteristics for a set of cell-free polynucleotides in the second sample mapped to the plurality of genomic regions, and the epigenetic rates of the plurality of genomic regions of the first sample; combining the plurality of likelihoods for one of more the plurality of genomic regions to determine an overall posterior probability for the presence of the cancer in the subject; and comparing the overall posterior probability for the presence of the cancer in the subject with a predetermined threshold. Some of these embodiments also include classifying a subject (a) as positive for circulating tumor DNA (ctDNA), if the overall posterior probability for the presence of the cancer in the subject is greater than or equal to the predetermined threshold, or (b) as negative for ctDNA, if the overall posterior probability for the presence of the cancer in the subject is less than the predetermined threshold. In some embodiments, the methods and systems used for analyzing the epigenetic status may be found in International Patent Application No. PCT/US2020/035605, entitled “METHODS AND SYSTEMS FOR IMPROVING PATIENT MONITORING AFTER SURGERY,” filed Jun. 1, 2020, which is incorporated by reference.

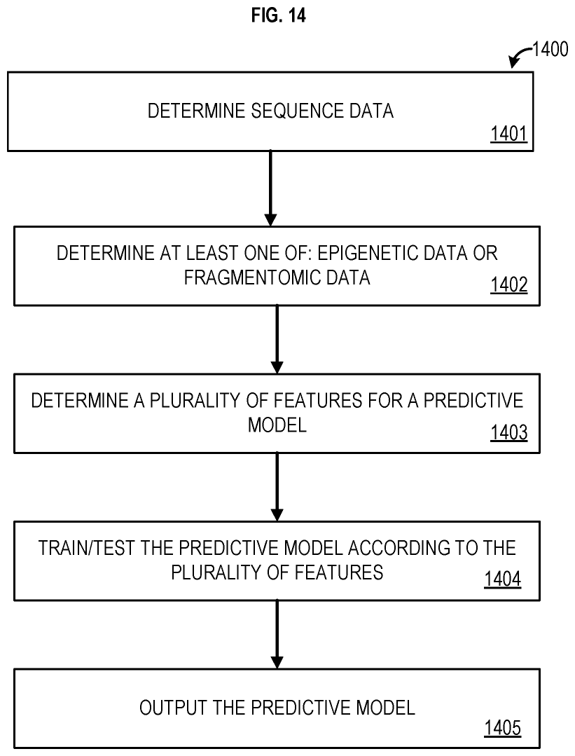

In an embodiment, shown in FIG. 14, a method 1400 for generating a predictive model is disclosed. In an embodiment, the sequence QC component 113, the epigenetic component 214, the fragmentomic component 215, the copy number component 216, the variant caller 217, additional components not shown (e.g., a component of the computer system 224) alone and/or in a combination thereof may be configured to access the sequence datastore 209 and/or the analysis datastore 218 and perform the method 1400 in whole and/or in part. The method 1400 may be performed in whole or in part by a single computing device, a plurality of computing devices, and the like. The method 1400 may comprise determining sequence data at 1401. The method 1400 may comprise determining at least one of: epigenetic data or fragmentomic data at 1402. The method 1400 may comprise determining a plurality of features for a predictive model at 1403. The method 1400 may comprise training and/or testing the predictive model according to the plurality of features at 1404. The method 1400 may comprise outputting the predictive model at 1405.

The plurality of genomic regions may comprise at least one of: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p161NK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET. Determining sequence data may comprise obtaining a plurality of samples from a plurality of subjects, wherein the plurality of samples comprise a plurality of cell-free nucleic acids. The plurality of genomic regions may comprise at least one of: a genomic region known to be associated with a cancer type, a genomic region associated with a known methylation status, a genomic region known to be associated with hypomethylation, or a genomic region known to be associated with therapy response.

The epigenetic data may comprise at least one of: information regarding DNA methylation, histone states or modifications, inflammation-mediated cytosine damage products, protein binding, or other molecular states reflected in the nucleic acid fragment analyzed that are not ascertained solely from the nucleotide base sequence, e.g., the methylation status of give base or set bases. Determining the epigenetic data associated with the plurality of sequence fragments comprises determining a methylation state of the plurality of sequence fragments.

Determining the methylation state of the plurality of sequence fragments may comprise determining at least one of: a methylation state vector or a methylated CpG density. Determining the methylation state vector may comprise aligning the plurality of sequence reads to a reference sequence, determining, based on the aligning, a methylation status of one or more CpG sites in a sequence read of the plurality of sequence reads and a location of the one or more CpG sites, and vectorizing the methylation status of the one or more CpG sites and the locations of the one or more CpG sites to generate the methylation state vector for the sequence read of the plurality of sequence reads. Determining the methylated CpG density may comprise aligning the plurality of sequence reads to a reference sequence, determining, based on the aligning, a methylation status of one or more CpG sites in a sequence read of the plurality of sequence reads, determining, based on the methylation status of the one or more CpG sites in the sequence read, that the sequence read is methylated or unmethylated, determining, for the plurality of sequence reads, a count of methylated sequence reads and a count of unmethylated sequence reads, and determining, based on the count of methylated sequence reads and the count of unmethylated sequence reads, the methylated CpG density.

The fragmentomic data may comprise at least one of: information regarding fragment size, nucleotide motifs at fragment ends, single-stranded jagged ends, genomic location of center point of the fragment length, genomic locations of fragment endpoints, genomic locations of fragment endpoints and/or any value indicating the endpoints of the fragment. Determining the fragmentomic data associated with the plurality of sequence fragments may comprise at least one of: determining a size of a sequence fragment of the plurality of fragments or determining an amount of the plurality of sequence fragments that have a particular size. The particular size may be a range. The range may be at least one of: 50-80, 50-100, 50-150, 100-150, 100-200, 150-200, 150-230, 200-300, or 300-400 bases.

Determining the fragmentomic data associated with the plurality of sequence fragments may comprise determining an end motif for the plurality of sequence fragments, wherein the end motif relates to an ending sequence of a sequence fragment. Determining the end motif for the plurality of sequence fragments may comprise aligning the plurality of sequence reads sequenced from the plurality of sequence fragments to a reference sequence and determining, based on the aligning, an end motif for each end of a sequence fragment of the plurality of sequence fragments. The ending sequences may comprise a number of bases, wherein the number of bases is between 1-6 bases. The ending sequences may comprise a number of bases that extends past the sequence fragment, wherein the number of bases is between 1-6 bases. The method 1400 may further comprise determining a frequency of occurrence of the end motif within the plurality of sequence fragments. The method 1400 may further comprise determining an end base of the end motif and determining a frequency of occurrence of the end base of the end motif. Determining the fragmentomic data associated with the plurality of sequence fragments may comprise determining a jagged end of a sequence fragment of the plurality of sequence fragments. Determining the jagged end of the sequence fragment of the plurality of sequence fragments may comprise determining an overhang index. The sequence fragment may be double-stranded with a first strand having a first portion and a second strand and determining the overhang index may comprise determining a methylation status of the first strand or the second strand that is proportional to a length of the first strand that overhangs the second strand and determining, based on the methylation status, the overhang index, wherein the overhang index provides a measure that a strand overhangs another strand.

Determining the fragmentomic data associated with the plurality of sequence fragments may comprise determining a genomic location of fragment endpoints. Determining the genomic location of fragment endpoints may comprise determining a windowed protection score (WPS). Determining the WPS may comprise determining a number of sequence fragments spanning a window and adjusting, based on any sequence fragments that start within the window, the number of sequence fragments spanning the window.

The method 1400 may further comprise determining an origin of a sequence fragment and assigning the origin of the sequence fragment to the sequence data, the epigenetic data, and the fragmentomic data associated with the sequence fragment. The origin may be tumor-derived or non-tumor derived, the origin is a tissue type, or the origin is a cancer type.

Determining, based on the at least the portion of the sequence data and the at least the portion of the at least one of: the epigenetic data or fragmentomic data, the plurality of features for the predictive model may comprise determining at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores and determining which, alone or in combination, of the at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores, have predictive value associated with an origin of a sequence fragment.

Training, based on the first portion of the sequence data and the at least one of: the epigenetic data or fragmentomic data, the predictive model according to the plurality of features may comprise training the predictive model according to a machine learning approach. The machine learning approach may comprise at least one of: a discriminant analysis, a decision tree, a nearest neighbor (NN) algorithm, a Bayesian network, a clustering algorithm, a neural network, a support vector machine (SVM), a logistic regression algorithm, a linear regression algorithms, a Markov model, or a principal component analysis (PCA). Testing, based on the second portion of the sequence data and the at least one of: the epigenetic data or fragmentomic data, the predictive model, may comprise causing the predictive model to be retrained.

The method 1400 may further comprise determining, for a subject, test sequence data comprising a plurality of sequence fragments associated with the plurality of genomic regions, wherein the plurality of sequence fragments are sequenced from a sample from the subject, determining at least one of: test epigenetic data or test fragmentomic data associated with the plurality of sequence fragments, providing, to the predictive model, test sequence data, test epigenetic data, and test fragmentomic data of the subject, and determining, based on the test sequence data, the test epigenetic data, and the test fragmentomic data of the subject, an origin of at least on sequence fragment in the sequence data. The origin may be one of tumor derived or non-tumor derived.

The method 1400 may further comprise administering one or more therapies to the subject based on the origin being tumor derived. The therapies may comprise administering chemotherapy, administering radiation therapy, or performing surgery to resect all or a portion of a tumor. The therapies may comprise administering at least one of: ALECENSA®, ALUNBRIG®, BRAFTOVI®, ERBITUX®, GAVRETO™, GILOTRIF®, HERCEPTIN®, IRESSA®, KADCYLA®, KEYTRUDA®, LORBRENA®, LUMAKRAS™, LYNPARZA®, MEKINIST®, OPDIVO®, PERJETA®, PIQRAY®, RETEVMO™, ROZLYTREK™ RUBRACA®, TABRECTA™, TAFINLAR®, TAGRISSO®, TALZENNA®, TARCEVA®, TEPMETKO™, TYKERB®, VITRAKVI®, VIZIMPRO®, XALKORI®, YBREVANT™ YERVOY®, or ZYKADIA®.

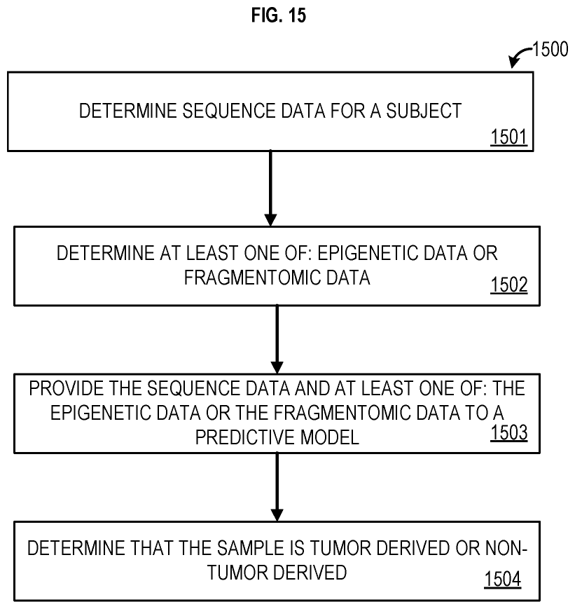

In an embodiment, shown in FIG. 15, a method 1500 for determining an origin of a sample is disclosed. In an embodiment, the sequence QC component 113, the epigenetic component 214, the fragmentomic component 215, the copy number component 216, the variant caller 217, additional components not shown (e.g., a component of the computer system 224) alone and/or in a combination thereof may be configured to access the sequence datastore 209 and/or the analysis datastore 218 and perform the method 1500 in whole and/or in part. The method 1500 may be performed in whole or in part by a single computing device, a plurality of computing devices, and the like. The method 1500 may comprise determining sequence data for a sample of a subject at 1501. The method 1500 may comprise determining at least one of: epigenetic data or fragmentomic data at 1502. The method 1500 may comprise providing the sequence data and at least one of the epigenetic data or the fragmentomic data to a predictive model. The method 1500 may comprise determining, based on the predictive model, that the sample is tumor derived or non-tumor derived. The method 1500 may further comprising generating the predictive model. Generating the predictive model may comprise determining sequence data of a plurality of sequence fragments associated with a plurality of genomic regions, wherein the sequence data comprises a plurality of sequence reads, wherein the plurality of sequence reads are sequenced from the plurality sequence fragments from a plurality of samples, wherein each sample of the plurality of samples is labeled as a tumor derived or a non-tumor derived, determining at least one of: epigenetic data or fragmentomic data associated with the plurality of sequence fragments, determining, based on at least a portion of the sequence data and at least a portion of at least one of: the epigenetic data or fragmentomic data, a plurality of features for the predictive model, training, based on a first portion of the sequence data and at least one of: the epigenetic data or fragmentomic data, the predictive model according to the plurality of features, testing, based on a second portion of the sequence data and at least one of: the epigenetic data or fragmentomic data, the predictive model, and outputting, based on the testing, the predictive model.

The plurality of genomic regions may comprise at least one of: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p161NK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET.

Determining sequence data may comprise obtaining a plurality of samples from a plurality of subjects, wherein the plurality of samples comprise a plurality of cell-free nucleic acids. The plurality of genomic regions may comprise at least one of: a genomic region known to be associated with a cancer type, a genomic region associated with a known methylation status, a genomic region known to be associated with hypomethylation, or a genomic region known to be associated with therapy response.

The epigenetic data may comprise at least one of: information regarding DNA methylation, histone states or modifications, inflammation-mediated cytosine damage products, protein binding, or other molecular states reflected in the nucleic acid fragment analyzed that are not ascertained solely from the nucleotide base sequence, e.g., the methylation status of give base or set bases. Determining the epigenetic data associated with the plurality of sequence fragments may comprise determining a methylation state of the plurality of sequence fragments. Determining the methylation state of the plurality of sequence fragments may comprise determining at least one of: a methylation state vector or a methylated CpG density. Determining the methylation state vector may comprise aligning the plurality of sequence reads to a reference sequence, determining, based on the aligning, a methylation status of one or more CpG sites in a sequence read of the plurality of sequence reads and a location of the one or more CpG sites, and vectorizing the methylation status of the one or more CpG sites and the locations of the one or more CpG sites to generate the methylation state vector for the sequence read of the plurality of sequence reads. Determining the methylated CpG density may comprise aligning the plurality of sequence reads to a reference sequence, determining, based on the aligning, a methylation status of one or more CpG sites in a sequence read of the plurality of sequence reads, determining, based on the methylation status of the one or more CpG sites in the sequence read, that the sequence read is methylated or unmethylated, determining, for the plurality of sequence reads, a count of methylated sequence reads and a count of unmethylated sequence reads, and determining, based on the count of methylated sequence reads and the count of unmethylated sequence reads, the methylated CpG density.

The fragmentomic data may comprise at least one of: information regarding fragment size, nucleotide motifs at fragment ends, single-stranded jagged ends, genomic location of center point of the fragment length, genomic locations of fragment endpoints, genomic locations of fragment endpoints and/or any value indicating the endpoints of the fragment. Determining the fragmentomic data associated with the plurality of sequence fragments may comprise at least one of: determining a size of a sequence fragment of the plurality of fragments or determining an amount of the plurality of sequence fragments that have a particular size. The particular size may be a range. The range may be at least one of: 50-80, 50-100, 50-150, 100-150, 100-200, 150-200, 150-230, 200-300, or 300-400 bases. Determining the fragmentomic data associated with the plurality of sequence fragments may comprise determining an end motif for the plurality of sequence fragments, wherein the end motif relates to an ending sequence of a sequence fragment. Determining the end motif for the plurality of sequence fragments may comprise aligning the plurality of sequence reads sequenced from the plurality of sequence fragments to a reference sequence and determining, based on the aligning, an end motif for each end of a sequence fragment of the plurality of sequence fragments. The ending sequence may comprise a number of bases. The number of bases may be between 1-6 bases. The ending sequence comprises a number of bases that extends past the sequence fragment, wherein the number of bases is between 1-6 bases. The method 1500 may further comprise determining a frequency of occurrence of the end motif within the plurality of sequence fragments. The method 1500 may further comprise determining an end base of the end motif and determining a frequency of occurrence of the end base of the end motif.

Determining the fragmentomic data associated with the plurality of sequence fragments comprises determining a jagged end of a sequence fragment of the plurality of sequence fragments. Determining the jagged end of the sequence fragment of the plurality of sequence fragments comprises determining an overhang index. The sequence fragment may be double-stranded with a first strand having a first portion and a second strand and determining the overhang index may comprise determining a methylation status of the first strand or the second strand that is proportional to a length of the first strand that overhangs the second strand and determining, based on the methylation status, the overhang index, wherein the overhang index provides a measure that a strand overhangs another strand.

Determining the fragmentomic data associated with the plurality of sequence fragments may comprise determining a genomic location of fragment endpoints. Determining the genomic location of fragment endpoints may comprise determining a windowed protection score (WPS). Determining the WPS may comprise determining a number of sequence fragments spanning a window and adjusting, based on any sequence fragments that start within the window, the number of sequence fragments spanning the window.

The method 1500 may further comprise determining an origin of a sequence fragment and assigning the origin of the sequence fragment to the sequence data, the epigenetic data, and the fragmentomic data associated with the sequence fragment. The origin may be tumor-derived or non-tumor derived, the origin is a tissue type, or the origin is a cancer type.

Determining, based on the at least the portion of the sequence data and the at least the portion of the at least one of: the epigenetic data or fragmentomic data, the plurality of features for the predictive model comprises determining at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores and determining which, alone or in combination, of the at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores, have predictive value associated with an origin of a sequence fragment.

Training, based on the first portion of the sequence data and the at least one of: the epigenetic data or fragmentomic data, the predictive model according to the plurality of features may comprise training the predictive model according to a machine learning approach. The machine learning approach may comprise at least one of: a discriminant analysis, a decision tree, a nearest neighbor (NN) algorithm, a Bayesian network, a clustering algorithm, a neural network, a support vector machine (SVM), a logistic regression algorithm, a linear regression algorithms, a Markov model, or a principal component analysis (PCA). Testing, based on the second portion of the sequence data and the at least one of: the epigenetic data or fragmentomic data, the predictive model, may comprise causing the predictive model to be retrained.

The method 1500 may further comprise, based on the sample being tumor derived, administering one or more therapies to the subject based on the origin being tumor derived. The therapies may comprise administering chemotherapy, administering radiation therapy, or performing surgery to resect all or a portion of a tumor. The therapies may comprise administering at least one of: ALECENSA®, ALUNBRIG®, BRAFTOVI®, ERBITUX®, GAVRETO™, GILOTRIF®, HERCEPTIN®, IRESSA®, KADCYLA®, KEYTRUDA®, LORBRENA®, LUMAKRAS™, LYNPARZA®, MEKINIST®, OPDIVO®, PERJETA®, PIQRAY®, RETEVMO™, ROZLYTREK™, RUBRACA®, TABRECTA™, TAFINLAR®, TAGRISSO®, TALZENNA®, TARCEVA®, TEPMETKO™, TYKERB®, VITRAKVI®, VIZIMPRO®, XALKORI®, YBREVANT™, YERVOY®, or ZYKADIA®.

### III. Cancer and Other Diseases

The present methods can be used to diagnose the presence or absence of conditions, particularly cancer, in a subject, to characterize conditions (e.g., staging cancer or determining heterogeneity of a cancer), select a treatment for a condition, monitor response to treatment of a condition, effect prognosis risk of developing a condition or subsequent course of a condition.

Various cancers may be detected using the present methods. Cancer cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as rare mutations. This phenomenon may be used to detect the presence or absence of cancer in individuals using the methods and systems described herein.

In certain embodiments, the methods and aspects disclosed herein are used to diagnose a given disease, disorder or condition in patients. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that may be evaluated using the methods and systems disclosed herein include DNA damage repair deficiency, achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

Cancers can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns.

Sequence data, epigenetic data, and/or fragmentomic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject, the method comprising generating a genetic profile of extracellular polynucleotides in the subject, wherein the genetic profile comprises a plurality of data resulting from copy number variation and rare mutation analyses. In some cases, including but not limited to cancer, a disease may be heterogeneous. Disease cells may not be identical. In the example of cancer, some tumors are known to comprise different types of tumor cells, some cells in different stages of the cancer. In other examples, heterogeneity may comprise multiple foci of disease. Again, in the example of cancer, there may be multiple tumor foci, perhaps where one or more foci are the result of metastases that have spread from a primary site.

### IV. Exemplary Precision Treatments

The precision diagnostics provided by the improved computer system 210 and/or 224 may result in precision treatment plans, which may be identified by the computer system 210 and/or 224 (and/or curated by health professionals). For example, one type of precision diagnostic and treatment may relate to genes in a pathway known to impact a specific cancer type.

The number and types of variant nucleotides in a sample can provide an indication of the amenability of the subject providing the sample to treatment, i.e., therapeutic intervention. For example, various poly ADP ribose polymerase (PARP) inhibitors have been shown to stop the growth of tumors from breast, ovarian and prostate cancers caused by hereditary mutations in the BRCA1 or BRCA2 genes.

The present analysis is also useful in determining the efficacy of a particular treatment option. Successful treatment options may increase the amount of copy number variation or rare mutations detected in a subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

In some embodiments, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain embodiments, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

### V. Systems and Computer Readable Media

The various processing operations and/or methods depicted in the Figures may be accomplished using some or all of the system components described in detail herein and, in some implementations, various operations may be performed in different sequences and various operations may be omitted. Additional operations may be performed along with some or all of the operations shown in the depicted flow diagrams. One or more operations may be performed simultaneously. Accordingly, the operations as illustrated (and described in greater detail herein) are provided as example and, as such, should not be viewed as limiting.

The present methods can be computer-implemented, such that any or all of the operations described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations.

Various operations of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like.

The present disclosure also includes an article of manufacture for analyzing a nucleic acid population that includes a machine-readable medium containing one or more programs which when executed implement the steps of the present methods.

The disclosure can be implemented in hardware and/or software. For example, different aspects of the disclosure can be implemented in either client-side logic or server-side logic. The disclosure or components thereof can be embodied in a fixed media program component containing logic instructions and/or data that when loaded into an appropriately configured computing device cause that device to perform according to the disclosure. A fixed media containing logic instructions can be delivered to a viewer on a fixed media for physically loading into a viewer's computer or a fixed media containing logic instructions may reside on a remote server that a viewer accesses through a communication medium to download a program component.

The present disclosure provides computer control systems that are programmed to implement methods of the disclosure. Returning to FIG. 2, the processor 220 may include a single core or multi core processor, or a plurality of processors for parallel processing. The storage device 222 may include random-access memory, read-only memory, flash memory, a hard disk, and/or other type of storage. The computer system 210 may include a communication interface (e.g., network adapter) for communicating with one or more other systems, and peripheral devices, such as cache, other memory, data storage and/or electronic display adapters. The components of the computer system 210 may communicate with one another through an internal communication bus, such as a motherboard. The storage device 222 may be a data storage unit (or data repository) for storing data. The computer system 210 may be operatively coupled to a network 223 (“network”) with the aid of the communication interface. The network 223 may be the Internet, an internet and/or extranet, or an intranet and/or extranet that is in communication with the Internet. The network 223 in some cases is a telecommunication and/or data network. The network 223 may include a local area network. The network 23 may include one or more computer servers, which can enable distributed computing, such as cloud computing. The network 223, in some cases with the aid of the computer system 210, may implement a peer-to-peer network, which may enable devices coupled to the computer system 220 to behave as a client or a server. The computer system 210 may exchange data with a computer system 224 using the network 223. For example, the computer system 224 may retrieve data from the analytics datastore 218.

The processor 220 may execute a sequence of machine-readable instructions, which can be embodied in a program or software. The instructions may be stored in a memory location, such as the storage device 222. The instructions can be directed to the processor 220, which can subsequently program or otherwise configure the processor 220 to implement methods of the present disclosure. Examples of operations performed by the processor 220 may include fetch, decode, execute, and writeback.

The processor 220 may be part of a circuit, such as an integrated circuit. One or more other components of the system 200 may be included in the circuit. In some cases, the circuit may include an application specific integrated circuit (ASIC).

The storage device 222 may store files, such as drivers, libraries and saved programs. The storage device 222 can store user data, e.g., user preferences and user programs. The computer system 210 in some cases may include one or more additional data storage units that are external to the computer system 210, such as located on a remote server that is in communication with the computer system 210 through an intranet or the Internet.

The computer system 210 can communicate with one or more remote computer systems through the network. For instance, the computer system 210 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 110 via the network.

Methods as described herein can be implemented by way of machine (e.g., computer processor) executable code stored on an electronic storage location of the computer system 210, such as, for example, on the storage device 222. The machine executable or machine readable code can be provided in the form of software (e.g., computer readable media). During use, the code can be executed by the processor 220. In some cases, the code can be retrieved from the storage device 222 and stored on the storage device 222 for ready access by the processor 220.

The code may be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system 210, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible, storage media, “media” may include other types of (intangible) media.

“Storage” media, terms such as computer or machine “readable medium” refer to any tangible (such as physical), non-transitory, medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 210 can include or be in communication with an electronic display 935 that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the processor 220.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

1. A method comprising:
determining sequence data of a plurality of sequence fragments associated with a plurality of genomic regions, wherein the sequence data comprises a plurality of sequence reads, wherein the plurality of sequence reads are sequenced from the plurality of sequence fragments from a plurality of samples, wherein each sample of the plurality of samples is labeled as a tumor derived or a non-tumor derived;
determining at least one of: epigenetic data or fragmentomic data associated with the plurality of sequence fragments;
determining, based on at least a portion of the sequence data and at least a portion of at least one of: the epigenetic data or fragmentomic data, a plurality of features for a predictive model;
training, based on a first portion of the sequence data and at least one of: the epigenetic data or fragmentomic data, the predictive model according to the plurality of features;
testing, based on a second portion of the sequence data and at least one of: the epigenetic data or fragmentomic data, the predictive model; and
outputting, based on the testing, the predictive model.

2. The method of claim 1, wherein the plurality of genomic regions comprises at least one of: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET.

3. The method of claim 1, wherein determining sequence data comprises obtaining a plurality of samples from a plurality of subjects, wherein the plurality of samples comprise a plurality of cell-free nucleic acids.

4. The method of claim 1, wherein the plurality of genomic regions comprise at least one of: a genomic region known to be associated with a cancer type, a genomic region associated with a known methylation status, a genomic region known to be associated with hypomethylation, or a genomic region known to be associated with therapy response.

5. The method of claim 1, wherein the epigenetic data comprises at least one of: information regarding DNA methylation, histone states or modifications, inflammation-mediated cytosine damage products, or protein binding.

6. The method of claim 1, wherein determining the epigenetic data associated with the plurality of sequence fragments comprises determining a methylation state of the plurality of sequence fragments.

7. The method of claim 5, wherein determining the methylation state of the plurality of sequence fragments comprises determining at least one of: a methylation state vector or a methylated CpG density.

8. The method of claim 7, wherein determining the methylation state vector comprises:
aligning the plurality of sequence reads to a reference sequence;
determining, based on the aligning, a methylation status of one or more CpG sites in a sequence read of the plurality of sequence reads and a location of the one or more CpG sites; and
vectorizing the methylation status of the one or more CpG sites and the locations of the one or more CpG sites to generate the methylation state vector for the sequence read of the plurality of sequence reads.

9. The method of claim 7, wherein determining the methylated CpG density comprises:
aligning the plurality of sequence reads to a reference sequence;
determining, based on the aligning, a methylation status of one or more CpG sites in a sequence read of the plurality of sequence reads;
determining, based on the methylation status of the one or more CpG sites in the sequence read, that the sequence read is methylated or unmethylated;
determining, for the plurality of sequence reads, a count of methylated sequence reads and a count of unmethylated sequence reads; and
determining, based on the count of methylated sequence reads and the count of unmethylated sequence reads, the methylated CpG density.

10.-25. (canceled)

26. The method of claim 1, further comprising determining an origin of a sequence fragment and assigning the origin of the sequence fragment to the sequence data, the epigenetic data, and the fragmentomic data associated with the sequence fragment.

27. The method of claim 26, wherein at least one of: the origin is tumor-derived or non-tumor derived, the origin is a tissue type, or the origin is a cancer type.

28. The method of claim 1, wherein determining, based on the at least the portion of the sequence data and the at least the portion of the at least one of: the epigenetic data or fragmentomic data, the plurality of features for the predictive model comprises:
determining at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores; and
determining which, alone or in combination, of the at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores, have predictive value associated with an origin of a sequence fragment.

29.-36. (canceled)

37. A method comprising:
determining, for a subject, sequence data of a plurality of sequence fragments associated with a plurality of genomic regions, wherein the sequence data comprises a plurality of sequence reads, wherein the plurality of sequence reads are sequenced from the plurality of sequence fragments from a sample from the subject;
determining at least one of: epigenetic data or fragmentomic data associated with the plurality of sequence fragments;
providing, to a trained predictive model, at least a portion of the sequence data and at least a portion of at least one of: the epigenetic data or the fragmentomic data; and
determining, based on the predictive model, that the sample is tumor-derived or non-tumor derived.

38. The method of claim 37, further comprising generating the predictive model.

39. The method of claim 38, wherein generating the predictive model comprises:
determining sequence data of a plurality of sequence fragments associated with a plurality of genomic regions, wherein the sequence data comprises a plurality of sequence reads, wherein the plurality of sequence reads are sequenced from the plurality sequence fragments from a plurality of samples, wherein each sample of the plurality of samples is labeled as a tumor derived or a non-tumor derived;
determining at least one of: epigenetic data or fragmentomic data associated with the plurality of sequence fragments;
determining, based on at least a portion of the sequence data and at least a portion of at least one of: the epigenetic data or fragmentomic data, a plurality of features for the predictive model;
training, based on a first portion of the sequence data and at least one of: the epigenetic data or fragmentomic data, the predictive model according to the plurality of features;
testing, based on a second portion of the sequence data and at least one of: the epigenetic data or fragmentomic data, the predictive model; and
outputting, based on the testing, the predictive model.

40. The method of claim 37, wherein the plurality of genomic regions comprises at least one of: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET.

41. The method of claim 39, wherein determining sequence data comprises obtaining a plurality of samples from a plurality of subjects, wherein the plurality of samples comprise a plurality of cell-free nucleic acids.

42. The method of claim 37, wherein the plurality of genomic regions comprise at least one of: a genomic region known to be associated with a cancer type, a genomic region associated with a known methylation status, a genomic region known to be associated with hypomethylation, or a genomic region known to be associated with therapy response.

43. The method of claim 37, wherein the epigenetic data comprises at least one of: information regarding DNA methylation, histone states or modifications, inflammation-mediated cytosine damage products, or protein binding.

44. The method of claim 37, wherein determining the epigenetic data associated with the plurality of sequence fragments comprises determining a methylation state of the plurality of sequence fragments.

45. The method of claim 44, wherein determining the methylation state of the plurality of sequence fragments comprises determining at least one of: a methylation state vector or a methylated CpG density.

46. The method of claim 45, wherein determining the methylation state vector comprises:
aligning the plurality of sequence reads to a reference sequence;
determining, based on the aligning, a methylation status of one or more CpG sites in a sequence read of the plurality of sequence reads and a location of the one or more CpG sites; and
vectorizing the methylation status of the one or more CpG sites and the locations of the one or more CpG sites to generate the methylation state vector for the sequence read of the plurality of sequence reads.

47. The method of claim 47, wherein determining the methylated CpG density comprises:
aligning the plurality of sequence reads to a reference sequence;
determining, based on the aligning, a methylation status of one or more CpG sites in a sequence read of the plurality of sequence reads;
determining, based on the methylation status of the one or more CpG sites in the sequence read, that the sequence read is methylated or unmethylated;
determining, for the plurality of sequence reads, a count of methylated sequence reads and a count of unmethylated sequence reads; and
determining, based on the count of methylated sequence reads and the count of unmethylated sequence reads, the methylated CpG density.

48. (canceled)

49. The method of claim 37, wherein determining the fragmentomic data associated with the plurality of sequence fragments comprises at least one of: determining a size of a sequence fragment of the plurality of fragments or determining an amount of the plurality of sequence fragments that have a particular size.

50.-72. (canceled)

73. A method of differentiating tumor and clonal hematopoiesis of indeterminate potential (CHIP) origin nucleic acid variants from one another in a test sample obtained from a test subject at least partially using a computer, the method comprising:
identifying, by the computer, test nucleic acid variants in a set of targeted genomic regions from sequence information obtained from nucleic acids in the test sample to produce a set of identified test nucleic acid variants;
identifying, by the computer, at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups;
matching, by the computer, given test nucleic acid variant-epigenetic signature groups in the set of test nucleic acid variant-epigenetic signature groups with reference nucleic acid variant-epigenetic signature groups corresponding to tumor origin nucleic acid variants or with reference nucleic acid variant-epigenetic signature groups corresponding to CHIP origin nucleic acid variants, thereby differentiating the tumor and the CHIP origin nucleic acid variants from one another in the test sample obtained from the test subject.

74.-116. (canceled)
